(19) United States
(12) Patent Application Publication
(10) Pub. No.: US 2006/0188459 A1
(43) Pub. Date: Aug. 24, 2006

(54) Cosmetic, pharmaceutical or dermatological preparations comprising copolymer waxes

(76) Inventors: Franz-Leo Heinrichs, Am Arenberg (DE); Anton Lukasch, Schleifweg (DE); Heike Michaelis, Am Hopfengarten (DE); Angela Lachmann, Hunsrueckstrasse (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/359,956

(22) Filed: Feb. 22, 2006

(30) Foreign Application Priority Data
Feb. 22, 2005 (DE) 10 2005 007 980.6

(51) Int. Cl. A61K 8/81

(52) U.S. Cl. 424/63; 424/70.11; 424/64; 424/70.8; 424/59; 424/66

(57) ABSTRACT

Cosmetic, pharmaceutical and dermatological preparations are described which contain copolymer waxes. The copolymer waxes contain structural units which are derived from α-olefins having 26 to 60 carbon atoms, from maleic anhydride, maleic acid or salts thereof and optionally from further monomers.

The present invention is described in the German priority application No. 102005007980.6, filed 22, Feb. 2005, which is hereby incorporated by reference as is fully disclosed herein.

The invention relates to cosmetic, pharmaceutical and dermatological preparations comprising copolymer waxes consisting of long-chain α-olefins and maleic anhydride, maleic acid or salts thereof.

Waxes and wax-like substances mainly determine the consistency of many cosmetic products. Waxes are used in order to influence the hardness and solidity of cosmetic products. The more hard waxes only melting at high temperatures are employed, the more solid the product.

Waxes are also used to provide active substances with protective coatings, to stabilize them, to delay their release but also to improve their compatibility with hydrophilic and lipophilic substances and thus the formulation ability. With the aid of waxes the water resistance of cosmetic products, for example sunscreen compositions, can additionally be increased.

In cosmetics, natural waxes of animal and vegetable origin, such as beeswax, berry wax, rose wax, Japan wax, China wax, shellac wax, quince wax, shea butter, candelilla wax, carnauba wax, lanolin (wool fat), jojoba oil and jojoba wax are often used. Because of pollution of natural waxes by pesticides, alternative, synthetically prepared waxes are sought which are free of plant protection agents and allergenic substances.

In DE 27 27 329, copolymers of α-olefins with maleic anhydride and, in DE 30 03 797, copolymers of C₃₀₊ olefins and maleic anhydride, and reaction products thereof with aliphatic monoalcohols are described, and the use thereof as lubricants in the metal and plastic processing industry is disclosed.

In WO 2004/041220 and WO 2004/041150, cosmetic preparations are described, in particular lipsticks and make-ups, comprising, in addition to a liquid fatty phase, a semicrystalline polymer of a C₁₄-C₂₄-α-olefin and a further monomer selected from carboxylic acid esters, preferably C₁₄-C₂₄-alkyl or C₁₁-C₁₅-perfluoroalkyl (meth)-acrylates, and N-alkyl(meth)acrylamides. Polymers of a C₁₄-C₂₄-α-olefin and a carboxylic anhydride, for example maleic anhydride are also mentioned. The polymers described are at least partially oil-soluble and are unsuitable for the production of temperature-stable sticks and pastes.

The object was to make available substances for cosmetic, pharmaceutical and dermatological preparations which have similarly good consistency-imparting properties as naturally occurring waxes, are readily compatible with aqueous systems and with oil systems, have a clear visual appearance, are easily processable and compatible with active substances (e.g. sunscreen filters), form water-repellent films on the skin, have temperature and storage stability, but are also skin-compatible and toxicologically harmless.

It has surprisingly been found that this object is achieved by copolymer waxes comprising structural units which are derived from α-olefins having more than 24 carbon atoms, maleic anhydride or maleic acid or salts thereof and optionally further monomers. It has been found that these copolymer waxes are easily emulsifiable because of high acid numbers, are suitable for the preparation of a wide variety of wax emulsions, are readily compatible with aqueous-based formulations, can be prepared in the form of water-in-wax dispersions but also readily thicken oils, exhibit excellent oil-binding power, are suitable as lubricants, dispersants and adhesives, and as polar synthetic waxes, they can be used as a substitute for natural waxes in cosmetic, pharmaceutical and dermatological preparations. Moreover, the copolymer waxes exhibit additional valuable application properties. They have a white to beige appearance, do not tend to form large crystallites, are soft and supple, readily processable and readily suitable for imparting consistency to pastes, creams, compacts and soft pencils; they are suitable for adjusting the viscosity of creamy emulsions or dispersions, and also hydroxy acid-containing and electrolyte-containing agents, improve the fine divisibility and stability of the emulsions and can be processed to give fluid preparations. They significantly improve the power of absorption of the pigments in the lipid phase and the pigment dispersion, and also the action of effect pigments.

Very advantageous for cosmetic use is also the cool sensation on the skin brought about by the copolymer waxes and the good spreading power, the water resistance, perspiration resistance, oil resistance and adhesiveness of the preparations containing them. The migration of solid ingredients (e.g. pigments) is suppressed, just as the tendency of individual ingredients to penetrate into the skin. A reduction in the irritant action of ingredients is thus achieved. Also advantageous is a delayed release of active substances, and an improved compatibility of individual components customary in cosmetic, pharmaceutical and dermatological preparations.

The invention relates to cosmetic, pharmaceutical and/or dermatological preparations which contain one or more copolymer waxes, comprising
a) one or more structural units —CH₂—CHR—, in which R is a linear or branched alkyl group having 24 to 58 carbon atoms,
b) optionally one or more structural units of the formulae (I)-(IV)

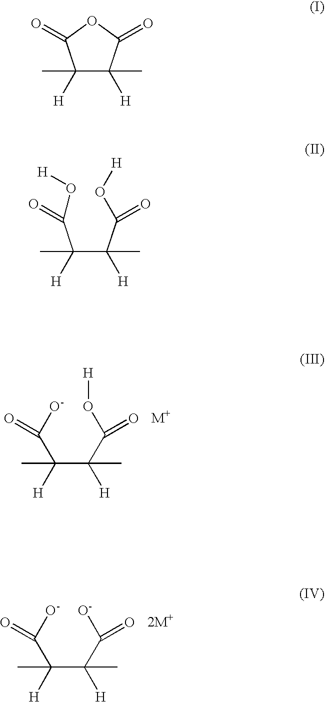

in which M⁺ are Li⁺, Na⁺, K⁺, Mg⁺⁺/2, Ca⁺⁺/2, Al⁺⁺⁺/3, NH₄⁺, monoalkylammonium, dialkylammonium, trialkylammonium and/or tetraalkylammonium ions, where the alkyl substituents of these ammonium ions in each case independently of one another can be (C₁-C₂₂)-alkyl radicals or (C₂-C₁₀)-hydroxyalkyl radicals, which can also be ethoxylated and where the 1 to 4 ethylene-oxide-containing radicals of these ammonium ions can in each case independently of one another contain from 2 to 10 ethylene oxide units and can have the same or a different degree of ethoxylation, and
c) optionally one or more structural units of the formulae

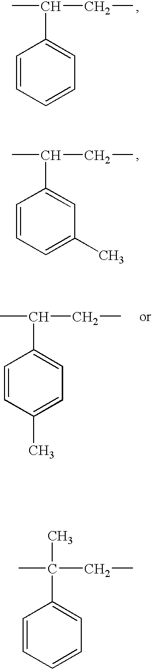

with the proviso that the structural units of components a), b) and optionally c) are arranged substantially alternately and the number of structural units of component a), of all structural units of component b) together and optionally of all structural units of component c) together is in each case in the range from 3 to 60.

In the copolymer waxes used in accordance with the invention it is also possible for there to be different M⁺.

The substantially alternate arrangement of the structural units of components a), b) and optionally c) means, for the purposes of the present invention, that the structural units of the respective components may be linked only to a certain proportion with another structural unit of the same component. For example, preferably 0 to 10 mol % and with particular preference 0 to 5 mol % of the structural units of component b) are linked directly to another structural unit of component b).

The structural units of component a) come about through polymerization of C₂₆-C₆₀ α-olefins.

The structural units of component b) come about through polymerization of maleic anhydride, which optionally after the polymerization is converted into the mono- or di-acid or into the corresponding salts.

The copolymer waxes used in accordance with the invention are comb polymers based on long-chain olefins and ethylenically unsaturated acids and their derivatives. The polar and apolar fractions are arranged substantially alternately. The mode of preparation of the copolymer waxes allows the molecular weight, crystallization behavior, viscosity, melting behavior and hardness to be influenced.

The viscosities are situated in the range from 100 mPas to 4000 mPas, preferably from 200 mPas to 2500 mPas, with particular preference from 250 mPas to 2000 mPas at 140° C., the melting points are situated in the range from 30° C. to 90° C., and the hardnesses, with needle penetration numbers (NPNs) of from 1 to 10, preferably from 2 to 6, with particular preference from 3 to 4 at 25° C., are situated in the middle range of known waxes. The principle of this characterization in accordance with the method ASTM D5—and NFT 004 consists in measuring the depth, expressed in tenths of a millimeter, to which a standardized needle, weighing 2.5 g and inserted in a needle carrier weighing 47.5 g, when placed on the wax, penetrates in 5 seconds at a given temperature.

In a preferred embodiment, the preparations according to the invention contain copolymer waxes which contain structural units of component a), in which R is a linear or branched alkyl group having 24 and/or 26 carbon atoms. These structural units of component a) are derived from C_{26/28}-α-olefins. Copolymer waxes having melting points of 30 to 80° C. are obtained, which exhibit good spreading power and good skin sensory properties. On application of these copolymer waxes to the skin or to the hair, water-repellent films result, which prevent the migration and washing out and washing off of ingredients of the cosmetic, pharmaceutical and dermatological compositions, in particular of active substances and/or pigments.

In a further preferred embodiment, the preparations according to the invention contain copolymer waxes which contain structural units of component a), in which R is a linear or branched alkyl group having 28 to 58 carbon atoms. These structural units of component a) are derived from C₃₀₊-α-olefins. Copolymer waxes having melting points of 35 to 75° C. are obtained, which are distinguished by good skin sensory properties. Moreover, harder waxes are obtained, which exhibit a very good oil-binding power and can be processed with the liquid lipid phase to give solid, temperature-resistant compositions, for example lipsticks.

In a further preferred embodiment, the preparations according to the invention contain waxes which contain structural units of component a), in which R is a linear or branched alkyl group
having 28 and/or 30 and/or 32 carbon atoms, or
having 30 and/or 32 and/or 34 carbon atoms, or
having 32 and/or 34 and/or 36 carbon atoms, or
having 34 and/or 36 and/or 38 carbon atoms, or
having 36 and/or 38 and/or 40 carbon atoms.

In a further preferred embodiment, the preparations according to the invention contain copolymer waxes which contain structural units of component a) and structural units of component b) according to formula (I).

In a further preferred embodiment, the preparations according to the invention contain copolymer waxes which contain structural units of component a) and structural units of component b) according to formula (II).

In a further preferred embodiment, the preparations according to the invention contain copolymer waxes which contain structural units of component a) and one or more structural units of component b) selected from formula (III) and formula (IV).

In one further preferred embodiment of the invention the copolymer waxes comprise components a) and b) in the component a):component b) molar ratios of 1:1-2, preferably of 1:1-1.1 and with particulr preference of 1:1.05.

In one particularly preferred embodiment, copolymer waxes are used which derive from C_{26/28} α-olefins and maleic anhydride, which are used in a molar ratio of 1:1-2, preferably 1:1-1.1, with particular preference 1:1.05, giving copolymer waxes having acid numbers of from 110 to 220, viscosities in the range of from 150 mPas to 4000 mPas at 140° C. and melting points in the range of from 75° C. to 90° C. The copolymer waxes are whitish-beige with a partially crystalline character.

In a further particularly preferred embodiment copolymer waxes are used which derive from C₃₀₊ α-olefins and maleic anhydride, which are used in a molar ratio of 1:1-2, preferably 1:1-1.1, with particular preference 1:1.05, giving copolymer waxes having acid numbers of from 85 to 170, viscosities in the range of from 100 mPas to 1500 mPas at 140° C. and melting points in the range of from 75° C. to 90° C. The copolymer waxes are whitish-beige with a crystalline character.

When the copolymer waxes are applied to the skin or hair, water-repellent (hydrophobic) films are formed which prevent the migration and the leaching or wash-off of ingredients of the cosmetic, pharmaceutical and dermatological preparations, in particular of the active substances and/or of the pigments.

The copolymers employed according to the invention have molecular weights preferably in the range from 1300 to 50 000, particularly preferably in the range from 5000 to 30 000 and especially preferably in the range from 10 000 to 20 000.

The copolymer waxes used according to the invention are prepared according to the methods described in DE 27 27 329, DE 30 03 797 and Chevron Chemical PA-18.

The copolymer waxes described above are of neutral odor, white to beige and have excellent processing properties; they are easily emulsifiable and therefore very highly suitable for the preparation of stable wax emulsions of the most varied type. Using the copolymer waxes together with oil components allows spreadable or flowable creams, cream foams or pastes to be prepared which have a viscosity of more than 500 mPas at 20° C. They are outstanding consistency-imparting agents, in particular for oil-based formulations and they possess good absorption properties, which can be utilized for the absorption of oils and the dispersion of pigments, odorants or solid active substances and other solid additives.

On account of their hardness, the copolymer waxes used according to the invention, e.g. lipsticks, kajal sticks and mascara sticks, impart stability even at relatively high temperatures. Particularly in the case of lipsticks, it is possible to utilize the high oil-binding power and the outstanding dispersing action. As a binder, the wax can be used together with lanolin, paraffin oil, isopropyl stearate, pigments and perfume for the production of eyeshadow, eyebrow pencils, powder compacts and rouge compacts. In this case, the water-repellent properties, and the thickening action of these copolymer waxes are utilized in order to suppress running of the fatty make-ups.

The copolymer waxes described above are suitable for the production of cosmetic, dermatological and pharmaceutical preparations, particularly advantageously for the production of decorative cosmetic compositions, sunscreen compositions, deodorants, hair care compositions and styling compositions, cleansing compositions for the skin, in particular peelings.

The preparations according to the invention can be the most different cosmetic, pharmaceutical and dermatological formulations. In particular, they can be cover sticks, acne sticks, lipsticks, make-ups, foundations, face powder, rouge, mascara, eyeshadow, eye-liner, peeling creams, pomades, hairstyling compositions, styling fluids, hair foams, hair gels, hair sprays, mousse, hair oil, tip fluids, hair tonics, night creams, care creams, nutrient creams, perfume creams, body lotions, ointments, lip care compositions, sunscreen compositions, deodorants, antiperspirants, colored gels in the form of pencils, such as e.g. multiphase pencils, sticks, pastes, powder, creams, cream foams, lotions, self-foaming, foamy, after-foaming or foamable emulsions, gels, roll-on preparations or foams.

A preferred embodiment is emulsions. The emulsions can be both water-in-oil emulsions and oil-in-water emulsions, microemulsions, nanoemulsions and multiple emulsions. The emulsions can be produced in a known manner, i.e. for example by means of cold, hot, hot/cold or PIT emulsification. A particularly preferred embodiment is self-foaming, foamy, after-foaming or foamable emulsions and microemulsions.

With the aid of emulsifiers, it is possible to produce a multiplicity of wax preparations. The selection of the emulsifier makes possible the production of nonionic and ionic wax dispersions.

The copolymer waxes employed according to the invention cause a good and finely divided power of absorption of pigments, solid active substances and solid additives in the oil phase and have a skin-smoothing and moisture-donating action.

The preparations according to the invention are distinguished by a particularly good adhesion of the cosmetic agents to the skin and form hydrophobic films which can barely be dissolved by developing cutaneous fat, so that undesired color shifts of the pigments and a migration of the active substances or solid additives do not occur.

The emulsions according to the invention contain at least

a) one of the copolymer waxes described above,

b) an oil component

c) an emulsifier

d) optionally further waxes.

The oil component can advantageously be selected from the groups consisting of the mineral oils, mineral waxes, oils, such as triglycerides, fats, waxes and other natural and synthetic fatty compounds, preferably esters of fatty acids with alcohols of low C number, e.g. with isopropanol, propylene glycol or glycerol, or esters of fatty alcohols with alkanoic acids of low C number with fatty acids or alkyl benzoates.

A class of preferred oils and fats and according to the invention are the triglycerides of linear or branched, saturated or unsaturated, optionally hydroxylated C₈-C₃₀-fatty acids, in particular vegetable oils, such as sunflower oil, corn oil, soybean oil, rice oil, jojoba oil, babasscu oil, pumpkin oil, grapeseed oil, sesame oil, walnut oil, apricot oil, orange oil, wheatgerm oil, peach kernel oil, macadamia oil, avocado oil, sweet almond oil, lady's smock oil, castor oil, olive oil, peanut oil, rapeseed oil and coconut oil, and also synthetic triglyceride oils, e.g. the commercial product Myritol®318. Hardened triglycerides are also preferred according to the invention. Oils of animal origin, for example bovine tallow, perhydrosqualene or lanolin, can also be employed.

A further class of preferred oils and fats according to the invention are the benzoic acid esters of linear or branched C₈₋₂₂-alkanols, e.g. the commercial products Finsolv®SB (isostearyl benzoate), Finsolv®TN (C₁₂-C₁₅-alkyl benzoate) and Finsolv®EB (ethylhexyl benzoate).

A further class of preferred oils and fats according to the invention are the dialkyl ethers having a total of 8 to 36 carbon atoms, in particular 12 to 24 carbon atoms, such as, for example, di-n-octyl ether (Cetiol® OE), di-n-decyl ether di-n-nonyl, ether, di-n-undecyl ether, di-n-dodecyl ether, di-3-ethyldecyl ether, tert-butyl n-octyl ether, isopentyl n-octyl ether, 2-methylpentyl n-octyl ether, n-hexyl n-octyl ether, n-octyl n-decyl ether, n-decyl n-undecyl ether, n-undecyl n-dodecyl ether and n-hexyl n-undecyl ether, and also di-tert-butyl ether or diisopentyl ether.

A further class of preferred oils and fats according to the invention are hydrocarbon oils, for example those having linear or branched, saturated or unsaturated C₇-C₄₀-carbon chains, for example petroleum jelly, dodecane, isododecane, cholesterol, lanolin, hydrogenated polyisobutylene, docosane, hexadecane, isohexadecane, paraffin oils, paraffin waxes, isoparaffin oils, e.g. the commercial products of the Permethyl® series, squalane, squalene, synthetic hydrocarbons such as polyisobutene and alicyclic hydrocarbons, e.g. the commercial product 1,3-di(2-ethylhexyl)cyclohexane (Cetiol®S), ozocerite, microwaxes and ceresin.

Likewise suitable are branched saturated or unsaturated fatty alcohols having 6-30 carbon atoms, e.g. isostearyl alcohol, and guerbet alcohols.

A further class of preferred oils and fats according to the invention are alkyl hydroxy-carboxylic ethers. Preferred alkyl hydroxycarboxylates are full esters of glycolic acid, lactic acid, malic acid, tartaric acid or citric acid. Further hydroxy-carboxylic acids which are suitable in principle are esters of β-hydroxypropionic acid, of tartronic acid, of D-gluconic acid, saccharic acid, mucic acid or glucuronic acid. Suitable alcohol components of these esters are primary, linear or branched aliphatic alcohols having 8 to 22 C atoms. In this case, esters of C₁₂-C₁₅-fatty alcohols are particularly preferred. Esters of this type are commercially obtainable, e.g. under the trade name Cosmacol® of EniChem, Augusta Industriale.

A further class of preferred oils and fats according to the invention are dicarboxylic acid esters of linear or branched C₂-C₁₀-alkanols, such as di-n-butyl adipate (Cetiol®B), di(2ethylhexyl) adipate, di(2-ethylhexyl) succinate, and diol esters such as ethylene glycol dioleate, ethylene glycol diisotridecanoate, propylene glycol di(2-ethylhexanoate), propylene glycol diisostearate, propylene glycol di-pelargonate, butanediol diisostearate and neopentyl glycol dicaprylate.

Likewise preferred oils and fats are symmetrical, unsymmetrical or cyclic esters of carbonic acid with fatty alcohols, glycerol carbonate or dicaprylyl carbonate (Cetiol®CC).

A further class of preferred oils and fats according to the invention are the esters of dimers of unsaturated C₁₂-C₂₂-fatty acids (dimer fatty acids) with monohydric linear, branched or cyclic C₂-C₁₈-alkanols or with polyhydric linear or branched C₂-C₆-alkanols.

In a further preferred embodiment of the invention, if the oil component is a silicone oil, the preparations according to the invention are preferably present in the form of a water-in-silicone emulsion and contain water, silicone, one or more emulsifiers and one or more copolymer waxes.

Silicone oils and waxes which are preferably available are dimethylpolysiloxanes and cyclomethicones, polydialkylsiloxanes R₃SiO(R₂SiO)ₓSiR₃, where R is methyl or ethyl, particularly preferably methyl, and x is a number from 2 to 500, for example the dimethicones obtainable under the trade names VICASIL (General Electric Company), DOW CORNING 200, DOW CORNING 225, DOW CORNING 200 (Dow Corning Corporation), and the dimethicones obtainable under SilCare® Silicone 41M65, SilCare® Silicone 41M70, SilCare® Silicone 41M80 (Clariant GmbH), stearyldimethylpolysiloxane, C₂₀-C₂₄-alkyl-dimethylpolysiloxane, C₂₄-C₂₈-alkyl-dimethylpolysiloxane, but also the methicones obtainable under SilCare® Silicone 41M40, SilCare® Silicone 41M50 (Clariant GmbH), furthermore trimethylsiloxy-silicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, for example the polymethylphenylsiloxanes obtainable under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty acid-, alcohol-, polyether-, epoxy-, fluoro- and/or alkyl-modified silicone compounds, and also polyethersiloxane copolymers.

Preparations present according to the invention as emulsions contain one or more emulsifiers from the group consisting of the nonionic, anionic, cationic or amphoteric emulsifiers.

Nonionic emulsifiers which are available are addition products of 0 to 30 mol of ethylene oxide and/or 0 to 5 mol of propylene oxide to linear fatty alcohols having 6 to 30 C atoms, preferably 10 to 22 C atoms, and very particularly preferably 14 to 22 C atoms. Those which can be employed are, for example, octanol (capryl alcohol), octenol, octadienol, decanol (capric alcohol), decenol, decadienol, dodecanol (lauryl alcohol), dodecadienol, ricinoleyl alcohol, myristyl alcohol, cetyl alcohol,-stearyl alcohol, cetearyl alcohol, oleyl alcohol, linoleyl alcohol, linolenyl alcohol, arachidyl alcohol, behenyl alcohol. Those which can also be employed are fatty alcohol cuts which are obtained by reduction of naturally occurring triglycerides, such as bovine tallow, palm oil, peanut oil, rapeseed oil, cottonseed oil, soybean oil, sunflower oil and linseed oil or which are produced from transesterification products using suitable alcohols of fatty acid esters and thus represent a mixture of different fatty alcohols. Such substances are commercially obtainable, for example, under the name Stenol®, e.g. Stenol® 1618, or Lanette®, e.g. Lanette® O and Lanette®22, or Lorol®, e.g. Lorol®C18.

Wool wax fats are also suitable.

Cetyl alcohol, stearyl alcohol, cetearyl alcohol, arachidyl alcohol and behenyl alcohol are particularly preferred.

A further class of preferred emulsifiers according to the invention are addition products of 0 to 30 mol of ethylene oxide and/or 0 to 5 mol of propylene oxide to linear and/or branched, saturated and/or unsaturated fatty acids having 6 to 30 C atoms, preferably 10 to 22 carbon atoms. Isostearic acid, such as the commercial products Emersol®871 and Emersol®875, isopalmitic acids such as Edenor®IP95, and all further fatty acids commercially available under the trade name Edenor® (Cognis) may be mentioned. Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeosteric acid, arachic acid, gadoleic acid, behenic acid, erucic acid and dimers of unsaturated fatty acids, and their technical mixtures. Fatty acid cuts from coconut oil or palm oil are particularly preferred. Stearic acid is especially preferred.

Customarily, the fatty acids are neutralized using a basic agent, e.g. NaOH, and used, for example, in the form of their sodium, potassium, ammonium, calcium, magnesium and zinc salts.

A further class of emulsifiers which can be used are esters of, if desired, alkylated sugars with C₆-C₃₀-fatty acids. Sugars which can be employed are any desired mono- or oligo-saccharides. Customarily, monosaccharides having 5 or 6 carbon atoms are employed, for example ribose, xylose, lyxose, altose, glucose, fructose, galactose, arabinose, altrose, mannose, gulose, idose, talose, and the deoxy sugars rhamnose and fucose. Sugars having 4 carbon atoms can also be employed, e.g. erythrose and threose. Oligosaccharides suitable according to the invention are composed of two to 10 monosaccharide units, e.g. sucrose (saccharose), lactose or trehalose. Preferred sugar units are the monosaccharides glucose, fructose, galactose, arabinose and the disaccharide sucrose. Glucose and sucrose are particularly preferred. The sugars can be partially etherified with methyl, ethyl, propyl, isopropyl or butyl groups, e.g. methyl glucoside, ethyl glucoside or butyl glucoside. For esterification, all C₆-C₃₀-fatty acids and their mixtures can be used which have been mentioned above. In principle, mono- and polyesterified sugars are suitable. The mono-, sesqui- and diesters are preferred, for example sucrose monostearate, sucrose distearate, sucrose monococoate, sucrose dicocoate, methyl glucoside monostearate, methyl glucoside sesquistearate, methyl glucoside isostearate, ethyl glucoside monolaurate, ethyl glucoside dilaurate, ethyl glucoside monococoate, ethyl glucoside dicocoate and butyl glucoside monococoate.

A further class of suitable emulsifiers are C₈-C₂₂-alkylmono- and -oligoglycosides, corresponding to the general formula RO—(Z)ₓ, where R is a C₈-C₂₂-alkyl group, Z is a sugar and x is the number of sugar units. The alkylmono- and -oligoglycosides which can be used according to the invention can only contain a certain alkyl radical R. Those alkylmono- and -oligoglycosides are particularly preferred in which R essentially consists of C₈- and C₁₀-alkyl groups, essentially of C₁₂- and C₁₄-alkyl groups, essentially of C₈- to C₁₆-alkyl groups or essentially of C₁₂- to C₁₆-alkyl groups. The sugar unit Z employed can be any desired mono- or oligosaccharide, such as have been mentioned above. Preferred sugar units are glucose, fructose, galactose, arabinose and sucrose, glucose being particularly preferred. The alkyl mono- and oligoglycosides which can be used according to the invention on average contain 1.1-5, preferably 1.1-2.0 and particularly preferably 1.1-1.8, sugar units.

The alkoxylated homologs of the alkyl mono- and oligoglycosides can also be employed according to the invention. Cocoyl glucoside, decyl glucoside, lauryl glucoside, cetearyl glucoside and arachidyl glucoside are suitable, for example.

In addition to the alkyl mono- and oligoglucosides mentioned, the mixtures of alkyl mono- and oligoglucosides and fatty alcohols, e.g. the commercially obtainable products Montanov®68 and Montanov®202, are also particularly preferred.

A further class of preferred emulsifiers are the partial esters of propylene glycol, glycerol and sorbitan mit C₈-C₂₂-fatty acids. For esterification, all C₈-C₂₂-fatty acids and their mixtures can be used which have already been mentioned above. Particularly suitable examples are propylene glycol monostearate, glycerol monolaurate, glycerol monostearate, glycerol distearate, glycerol monooleate, sorbitan monolaurate, sorbitan dilaurate, sorbitan monostearate, sorbitan sesquistearate, sorbitan distearate, sorbitan monoisostearate, sorbitan monooleate, sorbitan dioleate or the commercial products Monomuls®90-0, Monomuls®90-L 12 and Cutina®MD. These emulsifiers can on average contain up to 10 ethylene oxide and/or propylene oxide units per molecule.

A further preferred class of emulsifiers are polyglycerols of the formula HO—CH₂—CHOH—CH₂[—O—CH₂—CHOH—CH₂]ₙ—O—CH₂—CHOH—CH₂OH with n=0-8 and their esters with linear and branched C₈-C₂₂-fatty acids, which can carry functional groups in the alkyl chain, preferably polyglyceryl 2-dipolyhydroxystearate (commercial product Dehymuls® PGPH) and polyglyceryl 3-diisostearate (commercial product Lameform® TGI).

A further class of preferred emulsifiers are sterols, in particular cholesterol, lanosterol, β-sitosterol, stigmasterol, campesterol and ergosterol, and mycosterols. Customary commercial sterol emulsifiers are produced on the basis of soybean or rapeseed sterols. According to the invention, the employment of sterols which contain 5-10 ethylene oxide units per molecule is preferred. For example, the commercial products Generol®122, Generol® 122 E 5, Generol® 122 E 10 and Generol®RE-10 are suitable.

Emulsifiers which can likewise be employed are phospholipids, especially the phosphatidylcholines or lecithins. Phospholipids are phosphoric acid diesters, more rarely monoesters, of mostly linear saturated and unsaturated C₈-C₂₂-fatty acids. Soybean lecithin is preferred.

A further class of preferred emulsifiers are the esterification products of lactic acid or glycolic acid with linear or branched C₈-C₂₂-fatty acids, and the sodium, potassium, ammonium, calcium, magnesium and zinc salts of these esterification products.

Particularly preferred are esterification products of the general formula (5)

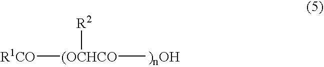

where R¹ is a linear or branched saturated or unsaturated alkyl radical having 5 to 21 carbon atoms and R² is a methyl group or a hydrogen atom and n is an integer from 1-4.

Among the acyl radicals R¹CO—, the radicals selected from the caproyl, capryloyl, caprinoyl, lauroyl, myristoyl, cetoyl, palmitoyl, stearoyl, isostearoyl and the oleyl group are in turn preferred. The stearoyl and the isostearoyl group are particularly preferred.

The radical R² is preferably methyl.

The degree of oligomerization n is preferably 1 or 2.

The compound sodium stearoyl-2-lactylate is especially preferred.

A further class of preferably employed emulsifiers are phosphoric acid mono-, di- and triesters of saturated or unsaturated linear or branched fatty alcohols having 8 to 30 carbon atoms and their ethylene oxide adducts having 1-10 ethylene oxide groups per molecule. These alkyl and alkenyl phosphates are shown in the general formula (6)

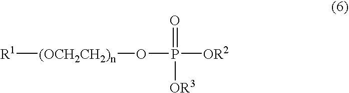

in which R¹ is a saturated or unsaturated, linear or branched hydrocarbon radical having 8 to 30 carbon atoms, R² and R³ independently of one another are a hydrogen atom, X or a radical (CH₂CH₂O)ₙR¹, n is numbers from 0 to 10 and X is an alkali metal or alkaline earth metal cation or a cation NR⁴R⁵R⁶R⁷, with R⁴ to R⁷ independently of one another being a C₁-C₄-hydrocarbon radical.

The alkyl and alkenyl phosphates preferred according to the invention contain, as a group R¹, alkyl radicals having 12-18 carbon atoms, which can be saturated or unsaturated and linear or branched. These groups R¹ are in particular lauryl, myristyl, cetyl, palmityl, stearyl, isostearyl and oleyl. Preferred values for n are either 0 or values from 1-10, preferably 2-5, particularly preferably 3-4 (alkyl or alkenyl ether phosphates). Furthermore, the use of ester mixtures of mono-, di- and tri- esters is preferred, where-the proportion of mono- and diesters predominates compared with the proportion of triester. The use of pure triesters can, however, also be preferred. Suitable commercial products are derived, for example, from the Hostaphat® series (Clariant), e.g. Hostaphat®KW 340 D, Hostaphat®KO300 N, Hostaphat®K0380 and Hostaphat®KL 340.

A further class of emulsifiers preferably employed according to the invention are acylglutamates of the formula (7)

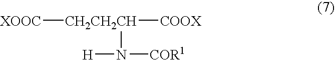

in which R¹CO is a linear or branched acyl radical having 6 to 22 carbon atoms and 0, 1, 2 or 3 double bonds and X is hydrogen, an alkali metal or alkaline earth metal cation, an ammonium, alkylammonium, alkanolammonium and/or glucammonium, for example acylglutamates which are derived from fatty acids having 6 to 22, preferably 12 to 18 carbon atoms, such as, for example, C_{12/14}- and/or C_{12/18}-coconut fatty acid, lauric acid, myristic acid, palmitic acid and/or stearic acid, in particular sodium N-cocoyl- and sodium N-stearoyl-L-glutamate.

A further class of emulsifiers preferred according to the invention are the esters of a hydroxy-substituted di- or tricarboxylic acid of the general formula (8),

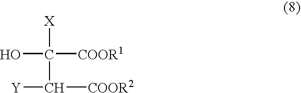

in which X is H or a —CH₂COOR group, Y is H or —OH, with the condition that Y is H if X is —CH₂COOR, R, R¹ and R² independently of one another are a hydrogen atom, an alkali metal or an alkaline earth metal cation, an ammonium group, the cation of an organoammonium base or a radical Z which is derived from a polyhydroxylated organic compound which is selected from the group consisting of the etherified (C₆-C₁₈)-alkylsaccharides having 1 to 6 monomeric saccharide units and/or the etherified aliphatic (C₆-C₁₆)-hydroxyalkylpolyols having 2 to 16 hydroxyl radicals, with the proviso that at least one of the groups R, R¹ or R² is a radical Z.

A further class of emulsifiers preferred according to the invention are the esters of the sulfosuccinic acid salt of the general formula (9)

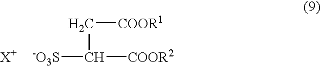

in which R¹ and R² independently of one another are a hydrogen atom, an alkali metal or alkaline earth metal cation, an ammonium group, the cation of an organo-ammonium base or a radical Z which is derived from a polyhydroxylated organic compound which is selected from the group consisting of the etherified (C₆-C₁₈)-alkylpolysaccharides having 1 to 6 monomeric saccharide units and/or the etherified aliphatic (C₆-C₁₈)-hydroxyalkylpolyols having 2 to 16 hydroxyl radicals, with the proviso that at least one of the groups R¹ or R² is a radical Z, and X⁺ is an alkali metal or alkaline earth metal cation, an ammonium group or the cation of an organo-ammonium base.

A further class of emulsifiers preferred according to the invention are the sulfosuccinic acid mono- and dialkyl esters having 8 to 24 C atoms in the alkyl group and sulfosuccinic acid monoalkylpolyoxyesters having 8 to 24 C atoms in the alkyl group and 1 to 6 ethoxy groups, and their alkali metal, alkaline earth metal or ammonium salts.

A further class of emulsifiers preferred according to the invention are the esters of tartaric acid and citric acid with alcohols, which are addition products of approximately 2 to 10 molecules of ethylene oxide and/or propylene oxide to fatty alcohols having 8 to 22 C atoms, and their alkali metal, alkaline earth metal or ammonium salts.

Further emulsifiers preferred according to the invention are ethercarboxylic acids of the formula R—O—(CH₂—CH₂O)ₓ—CH₂—COOH, in which R is a linear alkyl group having 8 to 30 C atoms and x is 0 or 1 to 10, acylsarcosinates having a linear or branched acyl radical having 6 to 22 carbon atoms and 0,1, 2 or 3 double bonds, acyltaurates having a linear or branched acyl radical having 6 to 22 carbon atoms and 0, 1, 2 or 3 double bonds, and acylisethionates having a linear or branched acyl radical having 6 to 22 carbon atoms and 0,1, 2 or 3 double bonds, and the alkali metal, alkaline earth metal or ammonium salts of these emulsifiers.

Amphoteric emulsifiers which are preferably available are alkylaminoalkylcarboxylic acids, betaines, sulfobetaines and imidazoline derivatives.

Preferably, fatty alcohol ethoxylates are chosen from the group consisting of the ethoxylated stearyl alcohols, cetyl alcohols, cetylstearyl alcohols, in particular polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether, polyethylene glycol(20) cetylstearyl ether.

Preferably, fatty acid ethoxylates are chosen from the group consisting of the ethoxylated stearates, isostearates and oleates, in particular polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

The ethoxylated alkyl ether carboxylic acid or its salts used can advantageously be sodium laureth(11EO) carboxylate.

An advantageous alkyl ether sulfate is lauryl diglycol ether sulfate sodium salt, an advantageous ethoxylated cholesterol derivative is polyethylene glycol(30) cholesteryl ether. Polyethylene glycol(25) soybean sterol is likewise preferred.

The ethoxylated triglycerides used can advantageously be polyethylene glycol(60) evening primose glycerides.

It is furthermore advantageous to choose the polyethylene glycol glycerol fatty acid esters from the group consisting of polyethylene glycol(20) glyceryllaurate, polyethylene glycol(6) glycerylcaproate/caprate, polyethylene glycol(20) glyceryloleate, polyethylene glycol(20) glycerylisostearate and polyethylene glycol(18) glyceryloleate/cocoate.

Among the sorbitan esters, polyethylene glycol (20)sorbitan monolaurate, polyethylene glycol (20)sorbitan monostearate, polyethylene glycol (20)sorbitan monoisostearate, polyethylene glycol (20)sorbitan monopalmitate, polyethylene glycol (20)sorbitan monooleate are particularly suitable.

Advantageous W/O emulsifiers which can be employed are: fatty alcohols having 8 to 30 carbon atoms, monoglycerol esters of saturated and/or unsaturated, branched and/or unbranched alkanecarboxylic acids of a chain length of from 8 to 24, in particular 12 to 18 C, atoms, diglycerol esters of saturated and/or unsaturated, branched and/or unbranched alkanecarboxylic acids of a chain length of from 8 to 24, in particular 12 to 18, C atoms, monoglycerol ethers of saturated and/or unsaturated, branched and/or unbranched alcohols of a chain length of from 8 to 24, in particular 12 to 18, C atoms, diglycerol ethers of saturated and/or unsaturated, branched and/or unbranched alcohols of a chain length of from 8 to 24, in particular 12 to 18, C atoms, propylene glycol esters of saturated and/or unsaturated, branched and/or unbranched alkanecarboxylic acids of a chain length of from 8 to 24, in particular 12 to 18, C atoms, and sorbitan esters of saturated and/or unsaturated, branched and/or unbranched alkanecarboxylic acids of a chain length of from 8 to 24, in particular 12 to 18, C atoms.

Especially advantageous W/O emulsifiers are glyceryl monostearate, glyceryl monoisostearate, glyceryl monomyristate, glyceryl monooleate, glyceryl monolaurate, glyceryl monocaprylate, glyceryl monocaprinate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate; saccharose distearate, cetyl alcohol, stearyl alcohol, arachidyl alcohol, behenyl alcohol, isobehenyl alcohol, selachyl alcohol, chimyl alcohol or polyethylene glycol(2) stearyl ether.

In the preparations according to the invention, mixtures of compounds from a number of these substance classes can be present.

In a further preferred embodiment, the preparations according to the invention are present in the form of oil-in-water emulsions.

In a particularly preferred embodiment, the preparations according to the invention are present as emulsions of the type oil-in-water, preferably as cosmetic or dermatological emulsions of the type oil-in-water, and contain, based on the total weight of the preparation,
a) up to 95% by weight, preferably 60 to 92% by weight, particularly preferably 70 to 90% by weight, especially preferably 75 to 85%. by weight, of a water phase,
b) up to 40% by weight, preferably 1 to 40% by weight, particularly preferably 2 to 25% by weight, especially preferably 5 to 20% by weight, of an oil phase,
c) up to 15% by weight, preferably 0.5 to 12% by weight, particularly preferably 1 to 8% by weight, especially preferably 1 to 5% by weight, of one or more emulsifiers and
d) up to 5% by weight, preferably 0.01 to 5% by weight, particularly preferably 0.05 to 3% by weight, especially preferably 0.1 to 2% by weight, of copolymer wax.

In a further particularly preferred embodiment, the preparations according to the invention are present as gel creams of the type oil-in-water, preferably as cosmetic or dermatological gel creams of the type oil-in-water, and contain, based on the total weight of the preparation,
a) up to 95% by weight, preferably 50 to 95% by weight, particularly preferably 70 to 90% by weight, especially preferably 75 to 85% by weight, of a water phase,
b) up to 30% by weight, preferably 1 to 30% by weight, particularly preferably 3 to 25% by weight, especially preferably 5 to 15% by weight, of an oil phase,
c) up to 5% by weight, preferably 0.5 to 5% by weight, particularly preferably 0.2 to 4% by weight, especially preferably 0.5 to 3% by weight, of one or more emulsifiers and
d) up to 5% by weight, preferably 0.01 to 5% by weight, particularly preferably 0.05 to 3% by weight, especially preferably 0.1 to 2% by weight, of copolymer wax.

In a further preferred embodiment, the preparations according to the invention are present in the form of water-in-oil emulsions.

In a particularly preferred embodiment, the preparations according to the invention are present as emulsions of the type water-in-oil, preferably as cosmetic or dermatological emulsions of the type water-in-oil, and contain, based on the total weight of the preparations,
a) up to 95% by weight, preferably 40 to 95% by weight, particularly preferably 50 to 90% by weight, especially preferably 60 to 85% by weight, of a water phase,
b) up to 60% by weight, preferably 2 to 60% by weight, particularly preferably 5 to 40% by weight, especially preferably 10 to 30% by weight, of an oil phase,
c) up to 20% by weight, preferably 0.5 to 20% by weight, particularly preferably 1 to 15% by weight, especially preferably 4 to 12% by weight, of one or more emulsifiers and
d) up to 5% by weight, preferably 0.01 to 5% by weight, particularly preferably 0.05 to 3% by weight, especially preferably 0.1 to 2% by weight, of copolymer wax.

In a further particularly preferred embodiment, the preparations according to the invention are present as water-in-silicone emulsions, preferably as cosmetic or dermatological water-in-silicone emulsions, and contain, based on the total weight of the preparations,
a) up to 90% by weight, preferably 20 to 90% by weight, particularly preferably 40 to 85% by weight, especially preferably 60 to 80% by weight, of a water phase,
b) up to 80% by weight, preferably 10 to 70% by weight, particularly preferably 20 to 60% by weight, especially preferably 30 to 50% by weight, of silicone oil,
c) 0.5 to 20% by weight, preferably 1 to 15% by weight, particularly preferably 3 to 10% by weight, of one or more emulsifiers and
d) 0.01 to 5% by weight, preferably 0.05 to 3% by weight, particularly preferably 0.1 to 2% by weight, of copolymer wax.

In a further preferred embodiment, the copolymer waxes employed according to the invention are in the form of micronized waxes, preferably in particle sizes of from 4 μm to 45 μm, preferably 4 μm to 20 μm. Micronized copolymer waxes consisting of very fine, rounded particles having a narrow particle size distribution are prepared from a wax melt in a spray process.

The micronized copolymer waxes can be more easily dispersed, cause better gliding properties of the compositions and improve the skin sensation and the dispersibility of the compositions on the skin and on the hair. The copolymer waxes described above can be incorporated particularly advantageously into peelings for the cleansing and care of the skin.

In a further preferred embodiment, the preparations according to the invention are present in the form of a dispersion and contain
a) a carrier material, preferably one or more oil components and/or solvents,
b) one or more emulsifiers and
c) one or more of the copolymer waxes described above and optionally one or more further waxes.

Wax dispersions, comprising
a) a carrier material, preferably one or more oil components and/or solvents,
b) one or more emulsifiers and
c) one or more of the copolymer waxes described above and optionally one or more further waxes

and in which the wax content is from 20 to 45% by weight, can also be used as a flowable preparation for incorporation into cosmetic, pharmaceutical and dermatological compositions.

The preparations according to the invention can, in addition to the copolymer waxes and/or micronized copolymer waxes, contain further waxes, such as, for example, polyethylene waxes, oxidized polyethylene waxes, amide waxes, carnauba waxes, montan waxes, paraffin waxes, Fischer-Tropsch waxes or polyvinyl waxes, optionally in combination with hydrophilic waxes, such as, for example, cetylstearyl alcohol.

In a further preferred embodiment, the preparations according to the invention are present as decorative compositions.

A further preferred embodiment is cosmetic and dermatological sticks, for example lipsticks, suncream sticks, antiacne sticks, eyebrow sticks, cover sticks and deo sticks, comprising
a) a lipid phase of at least one oil component and at least one copolymer wax, as described above,
b) substances optionally soluble or dispersible in the lipid phase,
c) an aqueous phase,
d) substances optionally soluble or dispersible in water,
e) optionally one or more active substances, and
f) at least one W/O emulsifier,

where the proportion of the aqueous phase, based on the finished preparation, can be 30 to 80% by weight.

In a further preferred embodiment, in particular if decorative compositions are concerned, the cosmetic and dermatological preparations according to the invention contain one or more colorants, preferably selected from color lakes, toners and pigments. In this context, they are preferably present in the form of powders, compacts, pastes, creams or sticks.

In a particularly preferred embodiment, the preparations according to the invention are present in the form of suspensions and contain, based on the total weight of the preparations,
a) 0.1 to 10% by weight, preferably 0.2 to 6% by weight, particularly preferably 0.3 to 5% by weight, of copolymer wax and
b) 0.1-30% by weight, preferably 0.5 to 15% by weight, particularly preferably 1.0 to 10% by weight, of solid particles, in particular selected from the group consisting of the dyes, color-imparting pigments, effect and light protection pigments, adsorbents and abrasive components.

In a further particularly preferred embodiment, the preparations according to the invention are present as gel-based eyeshadows and contain, based on the total weight of the preparations,
a) 0.1 to 10% by weight, preferably 0.2 to 6% by weight, particularly preferably 0.3 to 5% by weight, of copolymer wax and
b) 0.1 to 30% by weight, preferably 0.5 to 15% by weight, particularly preferably 1.0 to 10% by weight, of dyes and/or color-imparting pigments.

The preparations according to the invention can contain solid inorganic and organic particles. For decorative cosmetics, colored and also colorless pigments are employed. Some of the pigments mentioned below are also used as UV absorbers and/or light protection pigments.

The dyes and colored pigments, and both organic and inorganic dyes, can be selected from the appropriate positive list of the Cosmetics Act and/or from the EC list of cosmetic colorants. The following substances can be employed in the preparations according to the invention, for example.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Cerise Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfonic acid diethylamide-1′-phenylazo)-3- | 12490 | red |
| hydroxy-5″-chloro-2″,4″-dimethoxy-2-naphthoic acid anilide | | |
| Disperse Yellow 16 | 12700 | yellow |
| 1-(4-Sulfo-1-phenylazo)-4-aminobenzenesulfonic acid | 13015 | yellow |
| 2,4-Dihydroxyazobenzene-4′-sulfonic acid | 14270 | orange |
| 2-(2,4-Dimethylphenylazo-5-sulfonic acid)-1- | 14700 | red |
| hydroxynaphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonic acid-4-chloro-5-carboxylic acid-1-phenylazo)-2- | 15525 | red |
| hydroxynaphthalene | | |
| 1-(3-Methylphenylazo-4-sulfonic acid)-2-hydroxynaphthalene | 15580 | red |
| 1-(4′,(8′)-Sulfonic acid naphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2- | 15865 | red |
| hydroxynaphthalene-3-carboxylic acid | | |
| 1-(2-Sulfo-1-naphthylazo)-2-hydroxynaphthalene-3-carboxylic | 15880 | red |
| acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8,-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5- | 19140 | yellow |
| hydroxypyrazolone-3-carboxylic acid | | |
| Pigment Yellow 16 | 20040 | yellow |
| 2,6-(4′-Sulfo-2″,4″-dimethyl)bisphenylazo)-1,3- | 20170 | orange |
| dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″Sulfo-1″-phenylazo)-7′-sulfo-1′-naphthylazo]-1- | 27755 | black |
| hydroxy-7-aminonaphthalene-3,6-disulfonic acid | | |
| 4′-[(4″-Sulfo-1″-phenylazo)-7′-sulfo-1′-naphthylazo]-1- | 28440 | black |
| hydroxy-8-acetyl-aminonaphthalene-3,5-disulfonic acid | | |
| Direct Orange 34, 39, 44, 46, 60 | 40215 | orange |
| Food Yellow | 40800 | orange |
| trans-β-Apo-8′-Carotenaldehyde (C₃₀) | 40820 | orange |
| trans-Apo-8′-Carotenic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(diethylamino)triphenyl- | 42051 | blue |
| carbinol | | |
| 4-[(4-N-ethyl-p-sulfobenzylamino)phenyl-(4-hydroxy-2- | 42053 | green |
| sulfophenyl)(methylene)-1-(N-ethyl-N-p-sulfobenzyl)-2,5- | | |
| cyclohexadienimine] | | |
| Acid Blue 7 | 42080 | blue |
| (N-Ethyl-p-sulfobenzylaminophenyl-(2-sulfophenyl)- | 42090 | blue |
| methylene-(N-ethyl-N-p-sulfobenzyl)cyclohexadienimine | | |
| Acid Green 9 | 42100 | green |
| Diethyldisulfobenzyldi-4-amino-2-chlorodi-2-methylfuchsinimmonium | 42170 | green |
| Basic Violet 14 | 42510 | violet |
| Basic Violet 2 | 42520 | violet |
| 2′-Methyl-4′-(N-ethyl-N-m-sulfobenzyl)amino-4″-(N-diethyl)- | 42735 | blue |
| amino-2-methyl-N-ethyl-N-m-sulfobenzylfuchsinimmonium | | |
| 4′-(N-Dimethyl)amino-4″-(N-phenyl)aminonaphtho-N- | 44045 | blue |
| dimethylfuchsinimmonium | | |
| 2-Hydroxy-3,6-disulfo-4,4′-bis- | 44090 | green |
| dimethylaminonaphthofuchsinimmonium | | |
| Acid red | 45100 | red |
| 3,-(2′-Methylphenylamino)-6-(2′-methyl-4′- | 45190 | violet |
| sulfophenylamino)-9-(2″-carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenyl-aminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methyl-phenylamino)anthraquinone | 61565 | green |
| 1,4-Bis(o-sulfo-p-toluidine)anthraquinone | 61570 | green |
| Acid Blue 80 | 61585 | blue |
| Acid Blue 62 | 62045 | blue |
| N,N′-Dihydro-1,2,1′,2′-anthraquinoneazine | 69800 | blue |
| Vat Blue 6; Pigment Blue 64 | 69825 | blue |
| Vat Orange 7 | 71105 | orange |
| Indigo | 73000 | blue |
| Indigodisulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6,19; Natural Red 1 | 75100 | yellow |
| Bixin, Nor-Bixin | 75120 | orange |
| Lycopene | 75125 | yellow |
| trans-alpha, beta- or gamma-Carotene | 75130 | orange |
| Keto- and/or hydroxyl derivatives of carotene | 75135 | yellow |
| Guanine or pearl luster agents | 75170 | white |
| 1,7-Bis(4-hydroxy 3-methoxyphenyl)-1,6-heptadiene-3,5-dione | 75300 | yellow |
| Complex salt (Na, Al, Ca) of carminic acid | 75470 | red |
| Chlorophyll a and b; copper compounds of the chlorophylls | 75810 | green |
| and chlorophyllines | | |
| Aluminum | 77000 | white |
| Aluminum hydrate | 77002 | white |
| Water-containing aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromic oxide | 77288 | green |
| Chromic oxide, water-containing | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxides and hydroxides | 77491 | red |
| Hydrated iron oxide | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂.7H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)isoalloxazine, lactoflavin | | yellow |
| Caramel | | brown |
| Capsanthin, Capsorubin | | orange |
| Betanine | | red |
| Benzopyrilium salts, anthocyanines | | red |
| Aluminum, zinc, magnesium and calcium stearates | | white |
| Bromothymol Blue | | blue |
| Bromocresol Green | | green |
| Acid Red 195 | | red |

The preparations according to the invention can contain the dyes and pigments preferably in amounts from 0.1 to 30% by weight, particularly preferably in amounts from 0.5 to 15% by weight and in particular in amounts from 1.0 to 10% by weight, in each case based on the total weight of the preparations.

Preference is also given to the use of effect pigments. Effect pigments within the context of the present invention are understood as meaning pigments which due to their refraction properties produce special optical effects. Effect pigments impart to the treated surface (skin, hair, mucous membrane) luster or glitter effects or can visually conceal unevenness of the skin and skin wrinkles by means of diffuse light scattering. Particularly suitable effect pigments are, for example, mica particles which are coated with at least one metal oxide. In addition to mica, a layered silicate, silica gel and other SiO₂ modifications are also suitable as carriers. A metal oxide frequently used for coating is, for example, titanium oxide, to which, if desired, iron oxide, Cr₂, O₃, Prussian blue or carmine can be admixed. By means of the size and shape (e.g. spherical, ellipsoidal, flat, even, uneven) of the pigment particles and by means of the thickness of the oxide coating, the reflection properties can be influenced. Particularly preferred effects are pearl luster pigments, e.g. silver gray (guanine/hypoxanthine mixed crystals from fish scales) and mother of pearl (ground mussel shells), and monocrystalline pearl luster pigments such as, for example, bismuth oxychloride (BiOCl).

The effects can be controlled both by means of the particle size and by means of the particle size distribution of the pigment ensemble. Suitable particle size distributions extend, for example, from 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A wider particle size distribution, for example of 20-150 μm, produces glittering effects, whereas a narrower particle size distribution of <15 μm provides for a uniform silky appearance.

The preparations according to the invention contain effect pigments preferably in amounts from 0.1-20% by weight, particularly preferably 0.5-10 and especially preferably 1-5% by weight, in each case based on the total weight of the preparation.

The preferred inorganic light protection pigments are finely disperse or colloidally disperse metal oxides and metal salts, for example titanium oxide, zinc oxide, iron oxide, aluminum oxide, cerium oxide, zirconium oxide, silicates (talc) and barium sulfate. The particles should in this case have a mean diameter of less than 100 nm, preferably between 5 and 50 nm and particularly preferably between 15 and 30 nm, ‘nanopigments’. They can have a spherical form, but also those particles can be employed which have an ellipsoidal shape or one which differs in another way from the spherical shape. The pigments can also be surface-treated, i.e. be present in hydrophilized or hydrophobized form. Typical examples are coated titanium dioxides, such as, for example, titanium dioxide T 805 (Degussa) or Eusolex®T2000 (Merck). Possible hydrophobic coating agents are in this case especially silicones and in this context especially trialkoxyoctylsilanes or simethicones. Titanium dioxide and zinc oxide are particularly preferred.

The preferred inorganic particle substances are hydrophilic or amphiphilic. Advantageously, they can be superficially coated, in particular superficially water-repellent. Examples of these are titanium oxide pigments coated with aluminum stearate, zinc oxide coated with dimethylpolysiloxane (dimethicone), boron nitride coated with dimethicone and titanium oxide coated with a mixture of dimethylpolysiloxane and silica gel and hydrated aluminum oxide, titanium oxide coated with octylsilanol, or spherical polyalkylsesquioxane particles.

The preparations according to the invention can contain particulate inorganic or organic adsorbents having mean particle diameters of 1-100 μm. The adsorbents are selected from pyrogenic silicic acids, e.g. the aerosol types, precipitated silicic acids, silica gels, silicon dioxide, clays, e.g. bentonite or kaolin, magnesium aluminum silicates, e.g. talc and boron nitride, optionally modified starches and starch derivatives, cellulose powders, lactoglobulin derivatives, polymer powders of polyolefins, polycarbonates, polyurethanes, polyamides, polyesters, polystyrenes, polyacrylates, (meth)acrylate or (meth)acrylate-vinylidene copolymers, which can be crosslinked, Teflon or silicones, and mixtures of the substances mentioned.

The preparations according to the invention can contain abrasive components, for example ground plant parts such as almond bran or wheat bran, crystalline cellulose, hardened jojoba oil, polymer beads, preferably of polyethylene or polyamide-11, having mean diameters of 90-600 μm, and of active compound-containing micro- or millicapsules, the petrochemical polymers (e.g. from polyamide such as nylon-11) and/or biopolymers such as gelatin, pectin, plant gums, alginates and carrageenan. Almond bran, wheat bran, hardened jojoba oil and polyethylene beads are preferably employed.

In a further preferred embodiment of the invention, the preparations in question are make-up, eye make-up, mascara, eyeliner and rouge, distinguished by particular water resistance, color brilliance, pearl luster effect, good skin sensory properties and good dispersibility of the compositions on the skin.

In a further preferred embodiment of the invention, the preparations in question are nail varnish having excellent gloss effects.

A further preferred embodiment is peeling creams and gels for cleaning and smoothing the skin.

In a further preferred embodiment, the preparations according to the invention contain one or more UV light protection filters. These preparations are preferably sunscreens and are preferably present in the form of sprays, sticks, pastes, gels or lotions.

Suitable UV filters are preferably 4-aminobenzoic acid; 3-(4′-trimethyl-ammonium)benzylideneboran-2-one methylsulfate; 3,3,5-trimethylcyclohexyl salicylate; 2-hydroxy-4-methoxybenzophenone; 2-phenylbenzimidazole-5-sulfonic acid and its potassium, sodium and triethanolamine salts; 3,3′-(1,4-phenylenedimethine)bis(7,7-dimethyl-2-oxobicyclo-[2.2.1]-heptane-1-methanesulfonic acid and its salts; 1-(4-tert-butylphenyl)-3-(4-methoxyphenyl)propane-1,3-dione, 3-(4′-sulfo)benzylidenebornan-2-one and its salts; 2-ethylhexyl 2-cyano-3,3-diphenylacrylate; polymer of N-[2(and 4)-(2-oxoborn-3-ylidenemethyl)benzyl]acrylamide; 2-ethylhexyl 4-methoxycinnamate; ethoxylated ethyl 4-aminobenzoate; isoamyl 4-methoxycinnamate; 2,4,6-tris[p-(2-ethylhexyloxy-carbonyl)anilino]-1,3,5-triazine; 2-(2H-benzotriazol-2-yl)-4-methyl-6-(2-methyl-3-(1,3,3,3-tetramethyl-1-(trimethylsilyloxy)disiloxanyl)propyl)phenol; 4,4′-[(6-[4-((1,1-dimethylethyl)aminocarbonyl)phenylamino]-1,3,5-triazin-2,4-yl)diimino]bis(2-ethylhexyl benzoate); 3-(4′-methylbenzylidene)-D,L-camphor; 3-benzylidenecamphor; 2-ethylhexyl salicylate; 2-ethylhexyl 4-dimethylaminobenzoate; hydroxy-4-methoxybenzophenone-5-sulfonic acid (sulisobenzonum) and the sodium salt; and/or 4-isopropylbenzyl salicylate, N,N,N-trimethyl-4-(2-oxoborn-3-ylidenemethyl)anilium methylsulfate, homosalate (INN), oxybenzone (INN), 2-phenylbenzimidazole-5-sulfonic acid and their Na, K, and triethanolamine salts, alpha-(2-oxoborn-3-ylidene)-toluene-4-sulfonic acid and its salts, octyl methoxycinnate, isopentyl 4-methoxycinnamate, isoamyl p-methoxycinnamate, 2,4,6-trianilino-(p-carbo-2′-ethylhexyl-1′-oxy)-1,3,5-triazine(octyltriazone), phenol, 2-2(2H-benzotriazol-2-yl)-4-methyl-6-(2-methyl-3-(1,3,3,3-tetramethyl-1-(trimethylsilyl)oxy)disiloxanyl)propyl (drometriazole trisiloxane), benzoic acid, 4,4-((6-(((1,1-dimethylethyl)amino)-carbonyl)phenyl)amino)-1,3,5-triazine-2,4-diyl)diimino)bis, bis(2-ethylhexyl)) ester, 3-(4′-methylbenzylidene)-d-1-camphor (4-methylbenzylidene camphor), 3-benzylidene camphor, 2-ethylhexyl salicylate (octyl salicylate), ethyl-2-hexyl 4-dimethylaminobenzoate (octyl dimethyl PABA), 2-hydroxy-4-methoxybenzophenone-5-sulfonic acid (benzophenone-5) and the Na salt, 2,2′-methylenebis-6-(2H-benzotriazol-2yl)-4-(tetramethylbutyl)-1,1,3,3-phenol, sodium salt of 2-2′-bis(1,4-phenylene)-1H-benzimidazole-4,6-disulfonic acid, (1,3,5)-triazine-2,4-bis((4-(2-ethylhexyloxy)-2-hydroxy)phenyl)-6-(4-methoxyphenyl), 2-ethylhexyl 2-cyano-3,3-diphenyl-2-propenoate, glyceryl octanoate di-p-methoxycinnamic acid, p-aminobenzoic acid and ester, 4-tert-butyl-4′-methoxydibenzoylmethane, 4-(2-β-glucopyranoxy)propoxy-2-hydroxybenzophenone, octyl salicylate, methyl 2,5-diisopropylcinnamate, cinoxate, dihydroxydimethoxybenzophenone, disodium salt of 2,2′-dihydroxy-4,4′-dimethoxy-5,5′-disulfobenzophenone, dihydroxybenzophenone, 1-(3,4-dimethoxyphenyl)-4,4-dimethyl-1,3-pentanedione, 2-ethylhexyl dimethoxybenzylidene dioxoimidazolidine propionate, tetrahydroxybenzophenone, terephthalylidenedicamphorsulfonic acid, 2,4,6-tris[4-2-ethylhexyloxycarbonyl)anilino]-1,3,5-triazine, methyl bis(trimethylsiloxy)silyl isopentyl-trimethoxycinnamate, amyl p-dimethylaminobenzoate, 2-ethylhexyl p-dimethylaminobenzoate, isopropyl p-methoxycinnamate/diisopropyl cinnamate, 2-ethylhexyl p-methoxycinnamate, 2-hydroxy-4-methoxybenzophenone, 2-hydroxy-4-methoxybenzophenone-5-sulfonic acid and the trihydrate, 2-hydroxy-4-methoxybenzophenone-5-sulfonic acid, Na salt and phenylbenzimidazolesulfonic acid.

Further suitable UV filters are selected from the derivatives, which are solid at room temperature, of dibenzoylmethane, cinnamic acid esters, diphenic acid esters, benzophenone, camphor, p-aminobenzoic acid esters, o-aminobenzoic acid esters, salicylic acid esters, benzimidazoles, 1,3,5-triazines, monomeric and oligomeric 4,4-diarylbutadienecarboxylic acid esters and -carboxamides, ketotricyclo(5.2.1.0)decane, benzalmalonic acid esters, and any desired mixtures of the components mentioned. The organic UV filters can be oil-soluble or water-soluble. Particularly preferred oil-soluble UV filters according to the invention are 1-phenyl-3-(4′-isopropylphenyl)propane-1,3-dione, 2-octyl(dimethylamino)-benzoate, amyl 4-(dimethylamino)benzoate, 2-ethylhexyl 4-methoxycinnamate, propyl 4-methoxycinnamate, isopentyl 4-methoxycinnamate, 2-ethylhexyl 2-cyano-3,3-phenylcinnamate (octocrylene), 4-isopropylbenzyl salicylate, homomenthyl salicylate (3,3,5-trimethyloctylhexyl salicylate), 2-hydroxy-4-methoxybenzophenone, 2-hydroxy-4-methoxy-4′-methylbenzophenone, 2,2′-dihydroxy-4-methoxybenzophenone, di-2-ethylhexyl 4-methoxybenzmalonate, 2,4,6-trianilino(p-carbo-2′-ethyl-1′-hexyloxy)-1,3,5-triazine (octyltriazone) and dioctylbutamidotriazone, and any desired mixtures of the components mentioned.

Preferred water-soluble UV filters are 2-phenylbenzimidazole-5-sulfonic acid and its alkali metal, alkaline earth metal, ammonium, alkylammonium, alkanolammonium and glucammonium salts, sulfonic acid derivatives of benzophenones, preferably 2-hydroxy-4-methoxybenzophenone-5-sulfonic acid and its salts, sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and their salts.

The preparations according to the invention, for example the sunscreen compositions, contain UV light protection filters in the amounts by weight of preferably 0.1 to 10%, particularly preferably of 0.5 to 8% and especially preferably of 1 to 5%, based on the finished preparations.

The sunscreen compositions can also, however, contain as UV light protection filters the inorganic UV absorbers and light protection pigments mentioned under the abovementioned group of pigments, for example. The preparations according to the invention preferably contain these in amounts of 0.1 to 30% by weight, particularly preferably in amounts of 0.5 to 15% by weight and especially preferably in amounts of 1.0 to 10% by weight, in each case based on the total weight of the preparations.

In a further preferred embodiment, the preparations according to the invention contain, for example, the sunscreens and one or more antioxidants.

Advantageously, the antioxidants are chosen from the group consisting of amino acids (e.g. glycine, histidine, tyrosine, tryptophan) and their derivatives, imidazoles (e.g. urocaninic acid) and their derivatives, peptides such as D,L-carnosine, D-camosine, L-carnosine and their derivatives (e.g. anserine), carotenoids, carotenes (e.g. α-carotene, β-carotene, lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and its derivatives (esters, ethers, peptides, lipids, nucleotides, nucleosides and salts) and sulfoximine compounds (e.g. buthionine sulfoximine, homocysteine sulfoximine, buthionine sulfone, penta-, hexa-, heptathionine sulfoximine) in very low tolerable doses, furthermore (metal) chelators (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives, vitamin C and derivatives (e.g. ascorbylpalmitate, Mg ascorbylphosphate, ascorbylacetate), tocopherols and derivatives (e.g. vitamin E acetate), vitamin A and derivatives (vitamin A palmitate) and coniferyl benzoate of benzoin resin, rutic acid and its derivatives, α-glycosylrutin, ferulic acid, furfurylideneglucitol, carnosine, butylhydroxytoluene, butylhydroxyanisole, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenomethionine), stilbenes and their derivatives (e.g. stilbene oxide, trans-stilbene oxide), superoxide dismutase and the derivatives suitable according to the invention (salts, esters, ethers, sugars, nucleotides, nucleosides, peptides and lipids) of the substances mentioned.

Water-soluble antioxidants can be employed particularly advantageously within the meaning of the present invention.

The antioxidants are able to protect the skin and the hair from oxidative stress. Preferred antioxidants here are vitamin E and its derivatives and vitamin A and its derivatives.

The amount of the one or more antioxidants in the preparations according to the invention is preferably 0.001 to 30% by weight, particularly preferably 0.05 to 20% by weight and in particular 1 to 10% by weight, based on the total weight of the preparations.

If vitamin E and/or its derivatives is/are the antioxidant(s), it is advantageous to choose its/their respective concentrations from the range from 0.001 to 10% by weight, based on the total weight of the preparation. If vitamin A, or vitamin A derivatives, or carotenes or their derivatives is/are the antioxidant(s), it is advantageous to choose its/their respective concentrations from the range from 0.001 to 10% by weight, based on the total weight of the preparation.

In a particularly preferred embodiment of the invention, the cosmetic or pharmaceutical preparations contain antioxidants selected from superoxide dismutase, tocopherol (vitamin E) and ascorbic acid (vitamin C).

In a further preferred embodiment, the preparations according to the invention are deodorants and antiperspirants which contain one or more substances selected from substances having antimicrobial activity, astringents and deodorizing substances. These preparations are preferably present in the form of sprays, sticks, pastes, gels or lotions.

Preferably, suitable antimicrobial active compounds are cetyltrimethylammonium chloride, cetylpyridinium chloride, benzethonium chloride, diisobutylethoxyethyl-dimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy-metal salts, salicylates, piroctose, in particular zinc salts, pyrithione and its heavy-metal salts, in particular zinc pyrithione, zinc phenolsulfate, farnesol and combinations of these active substances.

The preparations according to the invention contain the antimicrobial active compounds preferably in amounts of up to 50% by weight, particularly preferably in amounts of from 0.01 to 10% by weight and especially preferably in amounts of from 0.1 to 10% by weight, based on the finished preparations.

Preferred astringents are oxides, preferably magnesium oxide, aluminum oxide, titanium dioxide, zirconium dioxide and zinc oxide, hydrated oxides, preferably hydrated aluminum oxide (boehmite) and hydroxides, preferably of calcium, magnesium, aluminum, titanium, zirconium or zinc.

The preparations according to the invention contain the astringent active compounds preferably in amounts of from 0 to 50% by weight, particularly preferably in amounts of from 0.01 to 10% by weight and especially preferably in amounts of from 0.1 to 10% by weight, based on the finished preparations.

Allantoin and bisabolol are preferred as deodorizing substances. These are preferably employed in amounts of from 0.0001 to 10% by weight.

In a further preferred embodiment, the preparations according to the invention are peelings. These are preferably present in the form of peeling creams or gels for cleansing and smoothing the skin.

As further excipients and additives, the preparations according to the invention can contain other pulverulent substances, filling materials, cationic polymers, film-forming agents, thickeners and dispersants, superfatting agents, moisturizing agents, stabilizers, biogenic active compounds, glycerol, preservatives, pearl luster agents, scents, solvents, opacifiers, further waxes, further protein derivatives such as gelatin, collagen hydrolyzates, natural and synthetic polypeptides, egg yolk, lecithin, lanolin and lanolin derivatives, fatty alcohols, silicones, cooling agents, for example methyl acetate, substances having keratolytic and keratoplastic action, enzymes and carrier substances.

In addition, the filling substances employed can be SiO₂, silica, ZnO, kaolin, SiO₂-modified kaolin, polytetrafluoroethylene, nylon, talc, mica, polymethyl methacrylate, polyethylene, polyethers, polycarbonates, polyvinyl chlorides, polystyrenes, polyamides, polyurethanes, polyacrylates, natural polymers, silk powder, microcrystalline cellulose, natural organic compounds such as encapsulated or unencapsulated grain meal and mixtures thereof.

The cationic polymers available are those known under the INCI name “Polyquaternium”, in particular polyquaternium-31, polyquaternium-16, polyquaternium-24, polyquaternium-7, polyquaternium-22, polyquaternium-39, polyquaternium-28, polyquaternium-2, polyquaternium-10, polyquaternium-11, as well as polyquaternium 37 & mineral oil & PPG trideceth (Salcare SC95), PVP-dimethylaminoethyl methacrylate copolymer, guar hydroxypropyltriammonium chlorides, and also calcium alginate and ammonium alginate. Cationic cellulose derivatives are likewise suitable; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethylenimines; cationic silicone polymers, such as, for example, amidomethicones; copolymers of adipic acid and dimethylaminohydroxypropyldiethylenetriamine; polyaminopolyamide and cationic chitin derivatives, such as, for example, chitosan.

Suitable silicone compounds are, for example, dimethylpolysiloxane, methylphenylpolysiloxanes, cyclic silicones and amino-, fatty acid-, alcohol-, polyether-, epoxy-, fluoro- and/or alkyl-modified silicone compounds, and also polyalkylsiloxanes, polyalkylarylsiloxanes, polyethersiloxane copolymers, such as described in U.S. Pat. No. 5,104,645 and the specifications cited therein, which can be present either in liquid or resinous form at room temperature.

Suitable film-forming agents are, depending on the intended use, salts of phenylbenzimidazolesulfonic acid, water-soluble polyurethanes, for example C₁₀-polycarbamyl-polyglycerol esters, but also polyvinyl alcohol, polyvinylpyrrolidone, and also copolymers, for example vinylpyrrolidone/vinyl acetate copolymer, water-soluble acrylic acid polymers/copolymers or their esters or salts, for example partial ester copolymers of acrylic/methacrylic acid and polyethylene glycol ethers of fatty alcohols, such as acrylate/steareth-20 methacrylate copolymer, water-soluble cellulose, for example hydroxymethylcellulose, hydroxyethylcellulose, hydroxypropylcellulose, water-soluble quaterniums, polyquaterniums, carboxyvinyl-polymers, such as carbomers and their salts, polysaccharides, for example polydextrose and glucan.

Superfatting agents which can be used are substances such as, for example, lanolin, lecithin, polyethoxylated lanolin derivatives, lecithin derivatives, polyol fatty acid esters, monoglycerides and fatty acid alkanolamides, where the latter can simultaneously serve as foam stabilizers.

Moisturizing substances available are, for example, isopropyl palmitate, glycerol and/or sorbitol, which are preferably employed in the amounts of 0.1 to 50% by weight.

Biogenic active compounds which can be employed are, for example, plant extracts, for example, Aloe vera, and vitamin complexes, Bisabolol®, Allantoin®, Phytantriol®, Panthenol®, AHA acids, local anesthetics, antibiotics, antiinflammatories, antiallergics, corticosteroids, sebostatics, phanthenol, allantoin and proteins.

Suitable preservatives are, for example, phenoxyethanol, diazolidinylurea, parabens, pentanediol, butylated hydroxytoluene, butylated hydroxyanisole or sorbic acid. They are preferably employed in the amounts of from 0.001 to 5% by weight, particularly preferably from 0.01 to 3% by weight and especially.preferably from 0.1 to 2% by weight, based on the finished preparations.

Colorants which can be used are the substances suitable and permitted for cosmetic and pharmaceutical purposes.

Substances preferably suitable as pearl luster-imparting components are fatty acid monoalkanolamides, fatty acid dialkanolamides, monoesters or diesters of alkylene glycols, in particular ethylene glycol and/or propylene glycol or its oligomers, with higher fatty acids, such as e.g. palmitic acid, stearic acid and behenic acid, monoesters or polyesters of glycerol with carboxylic acids, fatty acids and their metal salts, ketosulfones or mixtures of the compounds mentioned. Ethylene glycol distearates and/or polyethylene glycol distearates having on average 3 glycol units are particularly preferred.

If the preparations according to the invention contain pearl luster-imparting compounds, these are preferably contained in the preparations according to the invention in an amount of from 0.1 to 15% by weight and particularly preferably in an amount of from 1 to 10% by weight.

Suitable fungicidal active compounds are preferably ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn pyrithione and octopyrox.

Particularly suitable thickening agents and dispersants are ethylene glycol esters of fatty acids having 14 to 22, particularly preferably 16 to 22, carbon atoms, in particular mono- and diethylene glycol stearate. Likewise preferably suitable are stearic monoethanolamide, stearic diethanolamide, stearic isopropanolamide, stearic monoethanolamide stearate, stearyl stearate, cetyl palmitate, glyceryl stearate, stearamide diethanolamide distearate, stearamide monoethanolamide stearate, N,N-dihydrocarbyl-(C₁₂-C₂₂)-amidobenzoic acid and its soluble salts, N,N-dihydro-carbyl-(C₁₆-C₁₈)-amidobenzoic acid and its soluble salts and N,N-di(C₁₆-C₁₈)-amidobenzoic acid and its derivatives. Furthermore particularly suitable are polyacrylates and carbomers, in particular those water-soluble or water-swellable copolymers based on acrylamidoalkylsulfonic acids and N-vinylcarboxylic acid amides.

In order to increase the color intensity, the preparations according to the invention can contain the carriers customary in cosmetic systems, in particular benzyl alcohol, vanillin (4-hydroxy-3-methoxybenzaldehyde), isovanillin, p-hydroxyanisole, 3-hydroxy-4-methoxybenzaldehyde, 2-phenoxyethanol, salicylaldehyde, 3,5-di-hydroxybenzaldehyde, 3,4-dihydroxybenzaldehyde, 4-hydroxyphenylacetamide, methyl p-hydroxybenzoate, p-hydroxybenzaldehyde, m-cresol, hydroquinone monomethyl ether, o-fluorophenol, m-fluorophenol, p-fluorophenol, 2-(2′-hydroxy-phenoxy)ethanol, 3,4-methylenedioxyphenol, resorcinol monomethyl ether, 3,4-dimethoxyphenol, 3-trifluoromethylphenol, resorcinol monoacetate, ethylvanillin, 2-thiopheneethanol, butyl lacate and butyl glycolate. Preparations according to the invention comprising phenoxyethanol and/or benzyl alcohol are particularly advantageous with a synergistic action.

Suitable solubilizers are in principle all mono- or polyhydric alcohols and ethoxylated alcohols. Preferably, alcohols having 1 to 4 carbon atoms, such as, for example, ethanol, propanol, isopropanol, n-butanol and isobutanol, glycerol and their mixtures are employed. Furthermore preferred are polyethylene glycols having a relative molecular mass of below 2000. Particularly preferred are polyethylene glycols having a relative molecular mass of between 200 and 600 in amounts of up to 45% by weight and polyethylene glycols having a relative molecular mass of between 400 and 600 in amounts of from 0.5 to 15% by weight. Further suitable solvents are, for example, triacetin (glycerol triacetate) and 1-methoxy-2-propanol.

The stabilizers employed can be metal salts of fatty acids, such as, for example, magnesium stearate, aluminum stearate and/or zinc stearate.

The preparations according to the invention can be mixed with conventional ceramides, pseudoceramides, fatty acid N-alkylpolyhydroxyalkylamides, cholesterol, cholesterol fatty acid esters, fatty acids, triglycerides, cerebrosides, phospholipids and similar substances as care additives.

The scent or perfume oils used can be individual odorant compounds, e.g. the synthetic products of the type consisting of the esters, ethers, aldehydes, ketones, alcohols and hydrocarbons. Odorant compounds of the type consisting of the esters are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenyl glycinate, allylcyclohexyl propionate, styrallyl propionate and benzyl salicylate. The ethers include, for example, benzyl ethyl ether, the aldehydes, for example, the linear alkanals having 8 to 18 C atoms, citral, citronellal, citronellyloxyacetaldehyde, hydroxycitronellal, lilial and bourgeonal, the ketones, for example, the ionones, alpha-isomethylionone and methyl cedryl ketone, the alcohols anethol, citronellol, eugenol, geraniol, linalool, phenylethyl alcohol and terpineol, the hydrocarbons include mainly the terpenes and balsams. Preferably, mixtures of various odorants are used, which together produce a pleasant fragrance.

Perfume oils can also contain natural odorant mixtures, such as are accessible from vegetable or animal sources, e.g. pine, citrus, jasmine, lily, rose or ylang-ylang oil. Ethereal oils of relatively low volatility, which are usually used as flavoring components, are also suitable as perfume oils, e.g. sage oil, camomile oil, oil of cloves, melissa oil, mint oil, oil of cinnamon leaves, linden blossom oil, oil of juniper berries, vetiver oil, olibanum oil, galbanum oil and ladanum oil.

Further additives can be silicone compounds, preferably dimethylpolysiloxane, methylphenylpolysiloxanes, cyclic silicones and also amino-, fatty acid-, alcohol-, polyether-, epoxy-, fluoro- and/or alkyl-modified silicone compounds, for example alkylsilicones: SilCare® Silicone 41M10, SilCare® Silicone 41M15, SilCare® Silicone 41M20, SilCare® Silicone 41M30 (Clariant), alkyltrimethicones: SilCare® 31M30, SilCare® 31M40, SilCare® 31M50, SilCare® 31M60 (Clariant), phenyltrimethicones: SilCare® 15M30, SilCare® 15M40, SilCare® 15M50, SilCare® 15M60 (Clariant), polyalkylarylsiloxanes and polyethersiloxane copolymers.

The preparations according to the invention can contain the abovementioned silicone compounds preferably in the amounts by weight of from 0.1 to 20% by weight, particularly preferably from 0.2 to 15% by weight and especially preferably from 0.5 to 10% by weight, based on the finished preparations.

The preparations customarily have a pH in the range from 2 to 12 and preferably in a range from 3 to 8.

An outstanding property of the copolymer waxes employed according to the invention is their power of thickening oils.

The following examples and applications are intended to illustrate the invention in greater detail, but without restricting it thereto (all percentages are percentages by weight).

### EXAMPLE A

Copolymer wax 1

Copolymer of C₃₀₊-α-olefins with maleic anhydride

| Batch: | | |
|---|---|---|
| C₃₀₊-α-Olefin | 1680.00 | g (3.00 mol) |
| Maleic anhydride | 309.02 | g (3.15 mol) |
| Di-tert-butyl peroxide | 15.45 | g (5% by weight based |
| | | on maleic anhydride) |
| Synperonic LFRA 40 | 30.90 | g (10% by weight based |
| | | on maleic anhydride) |

Preparation:

The olefin is melted at 100° C., the batch is heated to 160° C., Synperonic LFRA 40 is added, and then di-tert-butyl peroxide in portions every 15 minutes. Following the first addition of peroxide, additionally, maleic anhydride is metered in portions under reflux every 15 minutes, and this is followed by stirring for 3 hours. The free monomers are removed under reduced pressure for 1 hour.

Acid number: 145

### EXAMPLE C

Copolymer wax 3

Copolymer of C_{26/28}-α-olefins with maleic anhydride

| Batch: | | |
|---|---|---|
| C_{26/28}-α-Olefin | 1196.80 | g (3.20 mol) |
| Maleic anhydride | 329.62 | g (3.36 mol) |
| Di-tert-butyl peroxide | 16.48 | g (5% by weight based |
| | | on maleic anhydride) |
| Synperonic LFRA 40 | 32.96 | g (10% by weight based |
| | | on maleic anhydride) |

Preparation:

The olefin is melted at 100° C., the batch is heated to 160° C., Synperonic LFRA 40 is added, and then di-tert-butyl peroxide in portions every 15 minutes. Following the first addition of peroxide, additionally, maleic anhydride is metered in portions under reflux every 15 minutes, and this is followed by stirring for 3 hours. The free monomers are removed under reduced pressure for 1 hour.

Acid number: 166.0

Chemical identification of the commercial products employed:

| | |
|---|---|
| Synperonic LFRA 40: | fatty alcohol ethoxylate/propoxylate |
| Measurement methods: | |
| Acid number mg KOH/g | ISO 2114 |

### FORMULATION EXAMPLES

### EXAMPLE 1

W/O cream

| | | | |
|---|---|---|---|
| A | Hostacerin ® DGI | | 4.00% |
| | Copolymer wax 3 | Clariant | 5.00% |
| | Magnesium stearate | | 1.00% |
| | Mineral oil, low viscosity | | 5.00% |
| | Petroleum jelly | | 10.00% |
| | Cetiol ® V | | 5.00% |
| B | 1,2-Propylene glycol | | 3.00% |
| | Water | | to 100% |
| | Preservative | | q.s. |
| C | Scent | | 0.40% |

Preparation:
I Melting of A at 80° C.
II Heating of B to 80° C.
III Stirring of II into I
IV Stirring until temperature of 35° C. is reached
V Addition of C to IV at 35° C.

### EXAMPLE 2

O/W cream

| | | | |
|---|---|---|---|
| A | Hostacerin ® DGI | | 2.00% |
| | SilCare ® Silicone 31M30 | Clariant | 4.00% |
| | Perliquidum | | 4.00% |
| | Copolymer wax 1 | Clariant | 2.00% |
| | Eutanol G | Clariant | 4.00% |
| | Isopropyl palmitate | Clariant | 4.00% |
| | Carbopol 980 | | 0.70% |
| B | Hostapon ® KCG | | 0.60% |
| | Sodium hydroxide solution (10% in water) | | 2.10% |
| | Preservative | | q.s. |
| | Scent | | 0.40% |
| | Dist. water | | to 100% |

Preparation:
I Heating of A to 80° C.
II Heating of B to 80° C.
III Emulsification by slowly stirring B into A.

### EXAMPLE 3

Hair tonic

| A | Water | to 100% |
|---|---|---|
| B | Genamin ® KSL | 7% |
| | Hostaphat ® KL 340 D | 1.5% |
| | Copolymer wax 1 | 1% |
| | Genapol ® PDB | 4% |
| | Jojoba oil | 1% |
| | Propylene glycol | 0.8% |
| | Isopropyl palmitate | 1% |
| | Dow Corning ® 190 | 0.8% |
| | Extrapon | 0.3% |
| | Vitamin E | 0.3% |
| | Panthenol (vitamin B5) | 0.5% |
| C | Citric acid | 0.2% |

Preparation:
I Heat A to 75° C.
II Heat B to 75° C.
III Add A to B with stirring and stir until cold
IV Adjust to pH 6 using C

### EXAMPLE 4

Cream rinse

| | | |
|---|---|---|
| A | Tylose ® H 100 000 YP2 | 1.5% |
| | Water, dist. | to 100% |
| B | Hostaphat ® KL 340 D | 1.5% |
| | Genapol ® PDB | 4% |
| | Copolymer wax 1 | 1% |
| C | Citric acid | 0.2% |

Preparation:
I Swell Tylose in the water at room temperature with stirring
II Heat I to 75° C.
III Heat B to 75° C.
IV Add II to III and stir until cold
V Adjust to pH 6 using C.

### EXAMPLE 5

Antiperspirant

| | | | |
|---|---|---|---|
| A | Locron ® L | Clariant | 10.00% |
| | Ethanol | | 50.00% |
| | Farnesol | | 0.50% |
| | Scent | | 0.20% |
| | Copolymer wax 1 | | 0.50% |
| | Water, dist. | | to 100% |
| | Extrapon Avocado special | | 1.50% |

Preparation:
Mixing of components A

### EXAMPLE 6

W/O antiperspirant cream

| | | |
|---|---|---|
| A | Abil EM90 | 2.0% |
| | Abil B8839 | 20.0% |
| | Copolymer wax 3 | 2.0% |
| B | Aloxicoll L | 17.0% |
| | Water, dist. | to 100% |
| | Perfume oil | q.s. |
| | Preservative | q.s. |

Preparation:
I Add phase B slowly to phase A with stirring at room temperature
II Homogenize

### EXAMPLE 7

Clear deo stick

| | | | |
|---|---|---|---|
| A | OCTOPIROX ® | (Clariant) | 0.10% |
| | Copolymer wax 3 | | 3% |
| | Propylene glycol | | 71.00% |
| | Rewoderm 66E | | 5.00% |
| | Sodium stearate | | 5.00% |
| | Genapol ® HS 020 | (Clariant) | 1.00% |
| | Water | | to 100% |

Preparation:
I Mixing of components A at about 50° C. and stirring until the solution is clear
II Filling and cooling

### EXAMPLE 8

Alcohol-free deodorant roll-on (opaque)

| | | |
|---|---|---|
| A | Tegodeo CW 90 | 2.0% |
| | Polyethylene glycol(3) lauryl ether | 1.0% |
| | Triethanolamine | 1.0% |
| B | Copolymer wax 1 | 1.2% |
| | Water, dist. | to 100% |
| C | Tagat R 40 | 3.0% |
| | Perfume oil | q.s. |
| | Preservative | q.s. |
| D | Citric acid (50% strength in water) | 0.2% |

Preparation:
I Heat phases A and B separately to 80° C.
II Stir phase B into phase A and homogenize
III Cool with slow stirring
IV Add phase C at 30° C.
V Adjust the pH with the aid of phase D

### EXAMPLE 9

Sunscreen gel

| A | Crodamol ® AB | | to 100% |
|---|---|---|---|
| | Neo Heliopan ® AV | | 7.50% |
| | SilCare ® Silicone 41M80 | (Clariant) | 5.00% |
| | Copolymer wax 3 | | 1% |
| B | Neo Heliopan ® BB | | 3.00% |

Preparation:
I Heat A to about 80° C.
II Dissolve B in A
III Fill and cool to 25° C. without stirring

### EXAMPLE 10

Foundation

| | | | |
|---|---|---|---|
| A | Nexbase ® 2004 FG | | 9.00% |
| | Myritol ® 318 | | 5.00% |
| | Almond Oil | | 4.00% |
| | SilCare ® Silicone 31M40 | (Clariant) | 4.00% |
| | SilCare ® Silicone 41M15 | (Clariant) | 3.00% |
| | Genapol ® HS020 | (Clariant) | 1.60% |
| | Genapol ® HS200 | (Clariant) | 2.40% |
| | Copolymer wax 1 | | 2.00% |
| B | Vanclay ® | | 1.50% |
| | Talc | | 3.00% |
| | Iron Oxide Pigments | | 7.90% |
| C | Glycerol | | 5.00% |
| | Water | | to 100% |
| | Aristoflex ® AVC | (Clariant) | 0.40% |
| D | Scent | | q.s. |
| | Nipaguard ® PDU | (Clariant) | q.s. |

Preparation:
I Mixing and melting of components A at about 70° C.
II Addition of B to I at about 70° C. with stirring
III Mixing of C until Aristoflex ® AVC is dissolved and heating to about 70° C.
IV Addition of C to II with stirring and homogenization
V Addition of D to IV at <40° C.

### EXAMPLE 11

Mascara

| | | | |
|---|---|---|---|
| A | Tylose ® H 4000 G4 | | 0.70% |
| | 1,2-Propylene glycol | | 1.00% |
| | Water | | to 100% |
| B | Triethanolamine 99% | | 1.20% |
| C | Stearic acid | | 3.00% |
| | SilCare ® Silicone 41M15 | (Clariant) | 1.00% |
| | SilCare ® Silicone 31M40 | (Clariant) | 2.00% |
| | Tegocare ® 450 | | 4.00% |
| | Nexbase ® 2006 | | 2.00% |
| | Beeswaxes | | 2.50% |
| | Candelilla wax | | 2.50% |
| | Copolymer wax 3 | | 3.50% |
| D | Pigments | | 10.00% |
| E | Nipagin ® M | (Clariant) | 0.20% |
| | Nipasol ® M | (Clariant) | 0.10% |
| F | Scent | | q.s. |

Preparation:
I Swell components A at room temperature with stirring; heat to 85° C.
II Addition of B to A and stirring
III Melting of components C at about 85° C.
IV Addition of D to III with stirring at 85° C.
V Addition of II to IV with vigorous stirring (about 15 minutes at 85° C., a further 15 minutes without heating)
VI Addition of E and F to V at about 35 to 40° C.
VII Filling at 35 to 40° C.

### EXAMPLE 12

Surfactant-free lotion with refreshing, invigorating action

| | | |
|---|---|---|
| A | Jojoba oil | 2.00% |
| | Almond oil | 3.00% |
| | Cetiol ® V | 3.00% |
| B | Copolymer wax 1 | 2.00% |
| C | Glycerol | 3.00% |
| | Menthol | 0.70% |
| | Camphor | 0.30% |
| | Ethanol | 5.00% |
| | Water | to 100% |
| | Preservative | q.s. |
| D | Scent | 0.30% |
| E | Citric acid (50% strength in water) | q.s. |

Preparation:
I Mix A and B
II Stir solution of C into I
III Add D to II
IV Homogenize
V Adjust pH to 6.00 with the aid of phase E.

INCI name of the commercial products employed:

| | | |
|---|---|---|
| Abil B8839 | | Cyclopentasiloxane/Cyclohexasiloxane |
| Abil EM90 | | Cetyldimethicone/Copolyol |
| Aloxicoll L. | | Aluminum Chlorohydrate |
| Aristoflex ® AVC | (Clariant) | Ammonium |
| | | Acryloyldimethyltaurate/VP |
| | | Copolymer |
| Carbopol 980 | | Polyacrylate |
| Cetiol ® V | (Cognis) | Decyl Oleate |
| Crodamol ® AB | | C₁₂₋₁₅ Alkyl Benzoate |
| Dow Corning ® 190 | (Dow Corning) | Dimethicone Copolyol |
| Euperlan ® PK 3000 | (Henkel) | Glycol Distearate, Laureth-4, |
| | | Cocamidopropyl Betaine |
| Eutanol G | | 2-Octyldodecanol |
| Extrapon ® | (Dragoco) | Plant Extracts |
| Extrapon Avocado special | | Water/Ethoxydiglycol/ |
| | | Propylene Glycol/Butylene |
| | | Glycol/Persea Gratissima Extract |
| Genamin ® CTAC | (Clariant) | Cetyltrimethylammonium Chloride |
| Genamin ® KDM-P | (Clariant) | Behenyltrimethylammonium Chloride |
| Genamin ® KSL | (Clariant) | PEG-5 Stearyl Ammonium Lactate |
| Genamin ® STAC | (Clariant) | Stearyltrimethylammonium Chloride |
| GENAPOL ® HS 020 | (Clariant) | Steareth-2 |
| Genapol ® HS200 | (Clariant) | Steareth-20 |
| Genapol ® PDB | (Clariant) | Glycol Distearate, Laureth-4, |
| | | Cocamidopropyl Betaine |
| Hostacerin ® DGI | | Polyglyceryl 2-Sesquiisostearate |
| Hostacerin ® T-3 | | Ceteareth-3 |
| Hostaphat ® KL 340 D | | Trilaureth-4 Phosphate |
| Hostapon ® KCG | | Sodium Cocoylglutamate |
| Locron ® L | (Clariant) | Aluminum Chlorohydrate |
| Myritol ® 318 | | Capric/Caprylic triglyceride |
| Neo Heliopan ® AV | | Ethylhexyl Methoxycinnamate |
| Neo Heliopan ® BB | | Benzophenone-3 |
| Nexbase ® 2004 FG | | Hydrogenated Poly-1-Decene |
| Nexbase ® 2006 | | Poly-1-Decene |
| Nipagin ® M | (Clariant) | Methylparaben |
| NIPAGUARD ® CMB | (Clariant) | Triethylene Glycol/ |
| | | Benzyl Alcohol/Propylene Glycol |
| Nipaguard ® PDU | (Clariant) | Propylene Glycol (and) Diazolidinyl |
| | | Urea (and) Methylparaben (and) |
| | | Propylparaben |
| Nipasol ® M | (Clariant) | Propylparaben |
| OCTOPIROX ® | (Clariant) | Piroctone Olamine |
| Perliquidum | | Paraffin Oil |
| Rewoderm 66E | | Isostearate |
| SilCare ® Silicone 31M40 | (Clariant) | Caprylyl Trimethicone |
| SilCare ® Silicone 31M50 | (Clariant) | Caprylyltrimethicone |
| SilCare ® Silicone 41M15 | (Clariant) | Caprylylmethicone |
| SilCare ® Silicone 41M80 | (Clariant) | C₂₄₋₂₈ Alkyl Dimethicone |
| Tagat R40 | | PEG-40 Hydrogenated Castor oil |
| Tegocare ® 450 | | Polyglyceryl-3 Methylglucose |
| | | Distearate |
| Tegodeo CW 90 | | Zinc Ricinoleate/Tetrahydroxypropyl- |
| | | Ethylendiamine/Laureth-3/ |
| | | Propylene Glycol |
| Tylose ® H 100000 YP2 | | Hydroxyethylcellulose |
| Tylose ® H 4000 G4 | | Hydroxyethylcellulose |
| Vanclay ® | | Kaolin |

1. A cosmetic, pharmaceutical or dermatological preparation containing one or more copolymer waxes, comprising
a) one or more structural units —CH₂—CHR—, wherein R is a linear or branched alkyl group having 24 to 58 carbon atoms,
b) optionally one or more structural units of the formulae (I)-(IV) 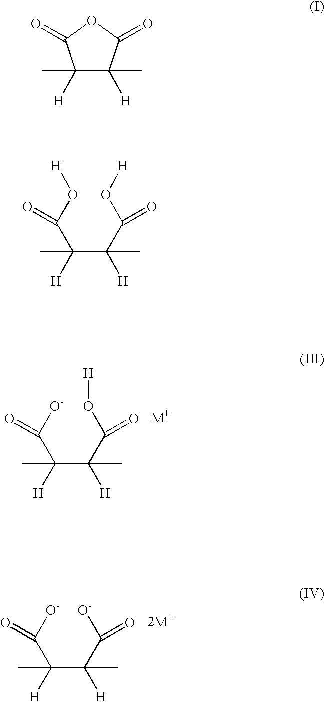
wherein M⁺ are Li⁺, Na⁺, K⁺, Mg⁺⁺/2, Ca⁺⁺/2, Al⁺⁺⁺/3, NH₄⁺, monoalkylammonium, dialkylammonium, trialkylammonium and/or tetraalkylammonium ions, where the alkyl substituents of the ammonium ions in each case independently of one another are (C₁-C₂₂)-alkyl radicals or (C₂-C₁₀)-hydroxyalkyl radicals, optionally ethoxylated and where the 1 to 4 ethylene-oxide-containing radicals of the ammonium ions in each case independently of one another optionally contain from 2 to 10 ethylene oxide units and have the same or a different degree of ethoxylation, and
c) optionally one or more structural units of the formulae 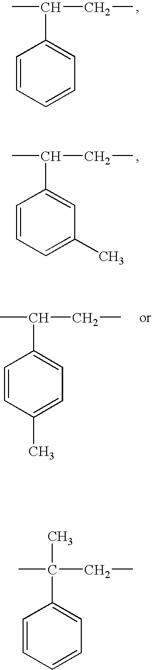
with the proviso that the structural units of components a), b) and optionally c) are arranged substantially alternately and the number of structural units of component a), of all structural units of component b) together and optionally of all structural units of component c) together is in the range from 3 to 60.

2. The preparation as claimed in claim 1, wherein the copolymer waxes contain structural units of component a), wherein R is a linear or branched alkyl group having 24 and/or 26 carbon atoms.

3. The preparation as claimed in claim 1, wherein the one or more copolymer waxes contain structural units of component a), wherein R is a linear or branched alkyl group having 28 to 58 carbon atoms.

4. The preparation as claimed in claim 1, wherein the one or more copolymer waxes contain structural units of component a) and structural units of component b) according to formula (I).

5. The preparation as claimed in claim 1, wherein the one or more copolymer waxes contain structural units of component a) and structural units of component b) according to formula (II).

6. The preparation as claimed in claim 1, wherein the one or more copolymer waxes contain structural units of component a) and one or more structural units of component b) selected from the group consisting of formula (III) and formula (IV).

7. The preparation as claimed in claim 1, wherein the one or more copolymer waxes contain the components a) and b) in the molar ratios component a):component b) of 1:1-2.

8. The preparation as claimed in claim 1, wherein the one or more copolymer waxes have molecular weights in the range from 1300 to 50 000.

9. A composition comprising the preparation as claimed in claim 1, wherein the composition is in a form selected from the group consisting of a cover stick, acne stick, lipstick, a make-up, a foundation, a face powder, rouge, a mascara, an eyeshadow, eye-liner, a peeling cream, pomade, hairstyling composition, styling fluid, a hair foam, a hair gel, hair spray, a mousse, a hair oil, a tip fluid, a hair tonic, night cream, care cream, nutrient cream, perfume cream, body lotion, ointment, a lip care composition, sunscreen composition, deodorant, antiperspirant, colored gel in the form of a pencil such as, for example, a multiphase pencil, a stick, a paste, a powder, a cream, a cream foam, a lotion, a self-foaming, foamy, after-foaming or foamable emulsion, a gel, roll-on preparation and a foam.

10. The preparation as claimed in claim 1, wherein the preparation is an emulsion.

11. The preparation as claimed in claim 10, wherein the emulsion is an oil-in-water emulsion.

12. The preparation as claimed in claim 11, comprising, based on the total weight of the preparation,
a) up to 95% by weight a water phase,
b) up to 40% by weight of an oil phase,
c) up to 15% by weight of one or more emulsifiers and
d) up to 5% by weight of the one or more copolymer waxes.

13. The preparation as claimed in claim 11, wherein the preparation is in the form of a gel cream and comprises, based on the total weight of the preparation,
a) up to 95% by weight of a water phase,
b) up to 30% by weight of an oil phase,
c) up to 5% by weight of one or more emulsifiers and
d) up to 5% by weight of the one or more copolymer waxes.

14. The preparation as claimed in claim 10, wherein the preparation is in the form of a water-in-oil emulsion.

15. The preparation as claimed in claim 14, comprising, based on the total weight of the preparation,
a) up to 95% by weight of a water phase,
b) up to 60% by weight of an oil phase,
c) up to 20% by weight of one or more emulsifiers and
d) up to 5% by weight of the one or more copolymer waxes.

16. The preparation as claimed in claim 1, wherein the one or more copolymer waxes are in micronized form.

17. The preparation as claimed in claim 1, wherein the preparation is the form of a dispersion and comprises
a) a carrier material,
b) one or more emulsifiers and
c) in addition to the one or more copolymer waxes optionally one or more further waxes.

18. The preparation as claimed in claim 1, wherein the Preparation is a decorative composition.

19. A composition comprising the preparation as claimed in claim 1, wherein the composition is in the form of a powder, compact, a paste cream or stick containing one or more colorants.

20. The preparation as claimed in claim 1, further comprising one or more UV light protection filters.

21. The preparation as claimed in claim 1, further comprising one or more antioxidants.

22. A deodorant or antiperspirant comprising one or more substances selected from substances having antimicrobial activity, astringents and deodorizing substances and the preparation as claimed in claim 1.

23. The deodorant or antiperspirant as claimed in claim 22, wherein the deodorizing substances are selected from allantoin and bisabolol.

24. A peeling comprising the preparation as claimed in claim 1.

25. The preparation as claimed in claim 1, wherein the one or more copolymer waxes contain the components a) and b) in the molar ratios component a):component b) of 1:1-1.1.

26. The preparation as claimed in claim 1, wherein the one or more copolymer waxes contain the components a) and b) in the molar ratios component a):component b) of 1:1.05.

27. The preparation as claimed in claim 1, wherein the one or more copolymer waxes have molecular weights in the range from 5000 to 30 000.

28. The preparation as claimed in claim 1, wherein the one or more copolymer waxes have molecular weights in the range from 10 000 to 20 000.

29. The preparation as claimed in claim 11, comprising, based on the total weight of the preparation,
a) from 60 to 92% by weight of a water phase,
b) from 1 to 40% by weight of an oil phase,
c) from 0.5 to 12% by weight of one or more emulsifiers and
d) from 0.01 to 5% by weight of the one or more copolymer waxes.

30. The preparation as claimed in claim 11, comprising, based on the total weight of the preparation,
a) from 70 to 90% by weight of a water phase,
b) from 2 to 25% by weight of an oil phase,
c) from 1 to 8% by weight of one or more emulsifiers and
d) from 0.05 to 3% by weight of the one or more copolymer waxes.

31. The preparation as claimed in claim 11, comprising, based on the total weight of the preparation,
a) from 75 to 85% by weight of a water phase,
b) from 5 to 20% by weight of an oil phase,
c) from 1 to 5% by weight of one or more emulsifiers and
d) from 0.1 to 2% by weight of the one or more copolymer waxes.

32. The preparation as claimed in claim 11, wherein the preparation is in the form of a gel cream and comprises, based on the total weight of the preparation,
a) from 50 to 95% by weight of a water phase,
b) from 1 to 30% by weight of an oil phase,
c) from 0.5 to 5% by weight of one or more emulsifiers, and
d) from 0.01 to 5% by weight of the one or more copolymer waxes.

33. The preparation as claimed in claim 11, wherein the preparation is in the form of a gel cream and comprises, based on-the total weight of the preparation,
a) from 70 to 90% by weight of a water phase,
b) from 3 to 25% by weight of an oil phase,
c) from 0.2 to 4% by weight of one or more emulsifiers, and
d) from 0.05 to 3% by weight of the one or more copolymer waxes.

34. The preparation as claimed in claim 11, wherein the preparation is in the form of a gel cream and comprises, based on the total weight of the preparation,
a) from 75 to 85% by weight of a water phase,
b) from 5 to 15% by weight of an oil phase,
c) from 0.5 to 3% by weight of one or more emulsifiers, and
d) from 0.1 to 2% by weight of the one or more copolymer waxes.

35. The preparation as claimed in claim 14, comprising, based on the total weight of the preparation,
a) from 40 to 95% by weight of a water phase,
b) from 2 to 60% by weight of an oil phase,
c) from 0.5 to 20% by weight of one or more emulsifiers, and
d) from 0.01 to 5% by weight of the one or more copolymer waxes.

36. The preparation as claimed in claim 14, comprising, based on the total weight of the preparation,
a) from 50 to 90% by weight of a water phase,
b) from 5 to 40% by weight of an oil phase,
c) from 1 to 15% by weight of one or more emulsifiers, and
d) from 0.05 to 3% by weight of the one or more copolymer waxes.

37. The preparation as claimed in claim 14, comprising, based on the total weight of the preparation,
a) from 60 to 85% by weight of a water phase,
b) from 10 to 30% by weight of an oil phase,
c) from 4 to 12% by weight of one or more emulsifiers, and
d) from 0.1 to 2% by weight of the one or more copolymer waxes.

38. The preparation as claimed in claim 17, wherein the carrier material is selected from the group consisting of an oil component, solvent or mixtures thereof.

39. The preparation as claimed in claim 19, wherein the one or more colorants is selected from the group consisting of color lakes, toners and pigments.

40. A sunscreen comprising the preparation as claimed in claim 20, wherein the sunscreen is in the form of a spray, stick, a paste, a gel or a lotion.

41. The deodorant or antiperspirant as claimed in claim 22, wherein the deodorant or antiperspirant is in the form of a spray, stick, paste, gel or lotion.
